(19) United States
(12) Patent Application Publication
(10) Pub. No.: US 2016/0082051 A1
(43) Pub. Date: Mar. 24, 2016

(54) USE OF SEA CUCUMBER GLYCOSAMINOGLYCAN IN PREPARING MEDICINE FOR PREVENTION AND TREATMENT OF THROMBOEMBOLIC DISEASE

(71) Applicants: SHANGHAI KAIRUN BIOLOGY MEDICINE LIMITED LIABILITY COMPANY, Shanghai (CN); HARBIN HONGDOUSHAN BIO-PHARM CO., LTD., Heilongjiang (CN)

(72) Inventors: Zhiguo Wang, Shangzhi (CN); Quanhai Liu, Shanghai (CN); Xuehai Wu, Shanghai (CN)

(73) Assignees: Shanghai Kairun Biology Medicine Limited Liability Company, Shanghai (CN); Harbin Hongdoushan Bio-Pharm Co., Ltd., Shangzhi, Heilongjiang (CN); Heilongjiang Hongdoushan Pharmaceutical Co., Ltd., Harbin, Heilongjiang (CN)

(21) Appl. No.: 14/890,854

(22) PCT Filed: May 7, 2014
(86) PCT No.: PCT/CN2014/000470

§ 371 Date: Nov. 12, 2015

(30) Foreign Application Priority Data
May 13, 2013 (CN) 201310175750.8

(51) Int. Cl. A61K 35/616; A61K 9/00; A61K 9/19; A61K 31/726

(52) U.S. Cl. CPC A61K 35/616; A61K 31/726; A61K 9/0019; A61K 9/19

(57) ABSTRACT

The present invention discloses use of sea cucumber glycosaminoglycan in the preparation of drugs. In particular, the present invention relates to use of medical use of sea cucumber glycosaminoglycan, and more particularly to use of depolymerized sea cucumber glycosaminoglycan or natural molecular segments of sea cucumber glycosaminoglycan with a weight average molecular weight greater than 54,500 Da in the preparation of a drug for the prevention and treatment of thromboembolic diseases. Thromboembolic diseases include atherosclerotic thrombotic diseases, venous thromboembolic diseases, hypercoagulable states and postoperative thrombosis or treatment of postoperative thrombi. The present invention has a wide treatment window for thromboembolic diseases, has a higher level of safety, and has good development and research value.

### TECHNICAL FIELD

The present invention relates to use of medical use of sea cucumber glycosaminoglycan, and more particularly to use of depolymerized sea cucumber glycosaminoglycan or natural molecular segments of sea cucumber glycosaminoglycan with a weight average molecular weight greater than 54,500 Da in the preparation of a drug for the prevention and treatment of thromboembolic diseases. Thromboembolic diseases include atherosclerotic thrombotic diseases, venous thromboembolic diseases, hypercoagulable states and postoperative thrombosis or treatment of postoperative thrombi.

### BACKGROUND ART

With the development of society and the population aging progress, the elderly are suffering from aging blood vessels and damaged walls of blood vessels, and are susceptible to high blood pressure, arteriosclerosis, and diabetes. After vascular endothelial cells are damaged, they generate increased thromboplastin, promote thrombinogenesis, as well as increase in thromboxanthin A2, and at the same time, the production of an anticoagulant substance prostacyclin is reduced, which will easily induce thrombosis. For example, when the blood glucose is increased, sugars bind to hemoglobin in erythrocytes, leading to tissue hypoxia all over the body. At this time, platelet agglutination is enhanced, and viscosity is increased, which easily promote thrombosis. Therefore, the incidence rates of thromboembolic diseases in middle-aged and elderly people are increasing with years. According to the statistics by the World Health Organization, there are 15 million people each year around the world who die from thrombotic diseases, i.e., local formation of blood coagulum, which is a leading cause resulting in arterial diseases such as myocardial infarction and stroke, as well as occurrence of venous thromboembolic diseases (including deep venous thrombosis and lung embolism), and patient death.

The drugs for the prevention and treatment of embolism formation can be divided into anticoagulant drugs, antiplatelet drugs, direct thrombolytic drugs and the like according to the mechanism of action, and can all be clinically used in the prevention and treatment of thrombotic diseases. The anticoagulant drugs prevent the thrombus formation or recurrence by affecting coagulation factors. The anticoagulant drugs have no dissolution effect on the formed thrombi but can prevent thrombus expansion and new thrombosis, which contributes to the autolysis of thrombi at an early stage. At the same time, the anticoagulant drugs have a significant preventive effect on venous thrombosis. Also, the anticoagulant drugs may also be used in cooperation with the prevention and treatment of thrombosis during extracorporeal circulation and hemodialysis, aiming to prevent blood coagulation during treatment operations.

The hypercoagulable state or thrombosis occurred in patients all involves many factors such as increased blood coagulating protein, activation of blood coagulating protein and increased blood coagulation. The clinical prevention and treatment principle for the hypercoagulative state includes, in addition to removal of cause of the hypercoagulative state, selection of a drug that reduces or inactivates the blood coagulating protein, to allow the hypercoagulative state to turn to a normal direction for development, and avoid the progress in the trend towards thrombus. Drugs for injection mainly include heparin and low molecular weight heparin, drugs for oral administration mainly include warfarin, dicoumarin, neodicoumarin and the like, and drugs for reducing blood viscosity to prevent and treat thromboembolism mainly include low molecular weight dextran. Warfarin is capable of inhibiting vitamin K dependent activation of some coagulation factors, and is now an anticoagulant drug with the maximal recipe quantity, and is, up to now, still clinically the only one orally effective vitamin K antagonist and the only one anticoagulant drug approved for long term application. Clinical researches have confirmed that, warfarin is capable of reducing the incidence rate of stroke by 64% in patients with auricular fibrillation. However, in spite of high efficacy, warfarin still brings on severe or even fatal bleeding risks. Moreover, because of great difference in pharmacokinetics among individuals and susceptibility to dietetic influences, as well as very complex drug interaction, it is difficult to administer warfarin at an optimal dose in the clinical practice. In addition, heparin and low molecular weight heparin drugs are susceptible to causing bleeding in clinical use, and people with different physiques need to be repeatedly detected in use. Therefore, all of the anticoagulant drugs clinically employed at present suffer from certain side effects. It is an inevitable trend, for the prevention and treatment of thromboembolic diseases, to screen and separate a more therapeutically effective and safer drug for prevention and treatment of thromboembolic diseases from traditional Chinese medicines, in consideration of the aging of the population and the increased incidence of thrombotic diseases, as well as the extensiveness of clinic applications of the existing anticoagulant drugs in the prevention and treatment of thromboembolic diseases and the seriousness of the potential safety hazards thereof.

### SUMMARY OF THE INVENTION

An objective of the present invention is to provide use of sea cucumber glycosaminoglycan in the preparation of a drug for the prevention and treatment of thromboembolic diseases, in order to overcome the above defects present in the prior art and meet the needs in clinical applications.

Animal experiments have proven that, for one or more segments of depolymerized sea cucumber glycosaminoglycan or natural molecular segments of sea cucumber glycosaminoglycan with a weight average molecular weight greater than 54,500 Da, the anticoagulant activity thereof assumes dose dependency. As compared with heparin and low molecular weight heparin, the blood coagulation effect thereof is increasing in an alleviating trend as the dose increases, and at the same dose, the onset time of efficacy is delayed as the weight average molecular weight increases, while the efficacy duration is increased. The natural molecular segments of sea cucumber glycosaminoglycan has an efficacy duration at a certain dose that may be up to 16 h.

Therefore, one or more segments of the depolymerized sea cucumber glycosaminoglycan or the natural molecular segments of sea cucumber glycosaminoglycan with a weight average molecular weight greater than 54,500 Da may be used in the preparation of a drug for the prevention and treatment of thromboembolic diseases. In particular, the thromboembolic diseases include atherosclerotic thrombotic diseases, venous thromboembolic diseases, hypercoagulable states, and postoperative thrombosis, or treatment of postoperative thrombi.

The “depolymerized sea cucumber glycosaminoglycan or the natural molecular segments of sea cucumber glycosaminoglycan with a weight average molecular weight greater than 54,500 Da” refers to depolymerized sea cucumber glycosaminoglycan of any weight average molecular weight and natural molecular segments of sea cucumber glycosaminoglycan with a weight average molecular weight greater than 54,500 Da, or a multisegment mixture of the depolymerized sea cucumber glycosaminoglycan of any weight average molecular weight and natural molecular segments of sea cucumber glycosaminoglycan with a weight average molecular weight greater than 54,500 Da.

Preferably, the depolymerized sea cucumber glycosaminoglycan has a weight average molecular weight of:

any segment of 54,500 Da to 57,000 Da, 57,010 Da to 62,990 Da, 63,000 Da to 67,000 Da, 67,010 Da to 72,990 Da, 73,000 Da to 77,000 Da, 77,010 Da to 82,990 Da, 83,000 Da to 87,000 Da, 87,010 Da to 92,990 Da, 93,000 Da to 97,000 Da, 97,010 Da to 102,900 Da, 103,000 Da to 107,000 Da, 107,010 Da to 112,990 Da, 113,000 Da to 117,900 Da, or 118,000 Da to 122,050 Da.

Particularly preferably, the depolymerized sea cucumber glycosaminoglycan has a weight average molecular weight of:

any segment of 54,500 Da to 57,000 Da, 58,000 Da to 62,000 Da, 63,000 Da to 67,000 Da, 68,000 Da to 72,000 Da, 73,000 Da to 77,000 Da, 78,000 Da to 82,000 Da, 83,000 Da to 87,000 Da, 88,000 Da to 92,000 Da, 93,000 Da to 97,000 Da, 98,000 Da to 102,000 Da, 103,000 Da to 107,000, 108,000 Da to 112,000 Da, 113,000 Da to 117,000 Da, or 118,000 Da to 122,000 Da.

The drug includes a therapeutically effective amount of the depolymerized sea cucumber glycosaminoglycan and a pharmaceutically acceptable carrier, and the pharmaceutically acceptable carrier is more than one selected from the group consisting of mannitol, lactose, dextran, glucose, glycine, hydrolyzed gelatin, povidone or sodium chloride, and preferably mannitol.

The drug is an injection solution for administration through intravenous or subcutaneous injection, or a lyophilized injection powder.

The depolymerized sea cucumber glycosaminoglycan or natural molecular segments of sea cucumber glycosaminoglycan with a molecular weight greater than 54,500 Da has a subcutaneous injection dosage of 1 mg/kg to 100 mg/kg, preferably 2 mg/kg to 80 mg/kg for rats; and an intravenous injection dosage of 0.1 mg/kg to 40 mg/kg, preferably 0.2 mg/kg to 30 mg/kg for rats.

In the drug, the depolymerized sea cucumber glycosaminoglycan has a purity of 90% to 99.99%, preferably more than 92%, and more preferably more than 94% for an ideal effect; and the natural molecular segments of sea cucumber glycosaminoglycan has a purity of 90% to 99.99%, preferably 92%, and more preferably 95% for an ideal effect.

The purity is purity by weight.

The depolymerized sea cucumber glycosaminoglycan has a polydispersity of 1 to 2, preferably 1 to 1.6, and more preferably 1 to 1.4.

The polydispersity refers to an index that measures the molecular weight distribution of polymers commonly used in the art, and is used for characterizing the width of molecular weight distribution of the polymers. The polydispersity is also called a polydispersity index, polydispersity or a distribution width index herein or in other literatures, and is a ratio of weight average molecular weight (Mw) to number average molecular weight (Mn), i.e. Mw/Mn. This ratio varies with the width of the molecular weight distribution. In single-dispersion, Mw/Mn is equal to 1, and the Mw/Mn value gradually increases as the molecular weight distribution widens.

The weight average molecular weight is defined as follows. The weight average molecular weight: all of synthetic high molecular compounds and most of natural high molecular compounds have non-uniform molecular weights, and they are mixtures of homologues with different molecular weights. In a polymer, a statistical average molecular weight by averaging the weights of molecules of different molecular weights is used.

The weight average molecular weight is tested by employing high performance liquid gel chromatography.

The depolymerized sea cucumber glycosaminoglycan may be a commercial product, e.g., the sea cucumber glycosaminoglycan or depolymerized sea cucumber glycosaminoglycan produced by Harbin Hongdoushan Bio-Pharm Co., Ltd., or may be prepared by employing a method as follows:

(1) an enzyme is added into minced sea cucumber, and then the mixture is subjected to enzymatic hydrolysis and precipitation, a crude product of sea cucumber glycosaminoglycan is collected, which is purified and decolorized, and depolymerized sea cucumber glycosaminoglycan is collected;

the sea cucumber is more than one selected from the group consisting of holothuria leucospilota, holothuria scabra, thelenota ananas, mensamaria intercedens or actinopyga mauritiana, preferably holothuria leucospilota or mensamaria intercedens;

the enzyme is a proteolytic enzyme and a compound pancreatin. The proteolytic enzyme may be a commercial product, for example, Alcalase produced by Novozymes (Shenyang) Biotechnology Co., Ltd., and the compound pancreatin may be a commercial product, for example, a compound pancreatin under a brand of Xuemei from Wuxi City Xuemei Enzyme Formulation Science and Technology Co., Ltd. The proteolytic enzyme is used in an amount that is 2% by weight of the sea cucumber, and the compound pancreatin is used in an amount that is 2 to 3% by weight of the sea cucumber;

(2) acetic acid at a concentration by weight of 5% and hydrogen peroxide at a concentration by weight of 3% are added into the product from step (1) for degradation, and depolymerized sea cucumber glycosaminoglycan with a weight average molecular weight greater than 54,5000 Da is collected;

the preparation method of the drug is a conventional method in the preparation field, such as a method recorded in the Chinese Medicine Preparation Manual, so as to obtain the injection solution or lyophilized injection powder;

(3) the sea cucumber glycosaminoglycan and depolymerized sea cucumber glycosaminoglycan with the desired molecular weight segments are collected for the segments with the desired molecular weight employing a gel column;

the gel adsorption column is, for example, a Sephadex-G100 gel adsorption column, a Sephadex-G50 gel adsorption column or a Sephadex-G200 gel adsorption column, and a dextran gel column from the U.S. GE Corporation may be employed as the Sephadex-G100 gel adsorption column.

The drug containing the depolymerized sea cucumber glycosaminoglycan according to the present invention can be applied to a patient in need of treatment by a subcutaneous or intravenous injection method, and the administration dose is determined by a physician according to the patient's specific circumstances (such as, age, weight, gender, disease duration, physical condition, and the like). Generally speaking, on the basis of the depolymerized sea cucumber glycosaminoglycan, the subcutaneous administration dose is 0.1 to 50 mg/kg, preferably 0.2 to 45 mg/kg, and the intravenous administration dose is 0.01 to 30 mg/kg, preferably 0.05 to 20 mg/kg.

Sea cucumber glycosaminoglycan is an acid mucopolysaccharide contained in the body wall of a sea cucumber, and is unique to sea cucumber. It is found in the present invention that, sea cucumber glycosaminoglycan has significant biological activities such as anticoagulation, anti-platelet aggregation, reduction of blood viscosity, fibrinolysis, adjustment of blood fat, and can be used in the treatment of thrombotic diseases.

Sea cucumber glycosaminoglycan is further depolymerized into segments of depolymerized sea cucumber glycosaminoglycans with different molecular weights, which exhibit different anticoagulant activity, and the anticoagulant activity thereof is increased progressively in an alleviating trend as the dose increases, which is safer than heparin drugs and vitamin K antagonist drugs. Sea cucumber glycosaminoglycan has a wide treatment window and high safety for clinically treating thromboembolic diseases, as well as a good value for development and research.

### BRIEF DESCRIPTION OF THE DRAWINGS

FIG. 1 is a purity diagram of sea cucumber glycosaminoglycan and depolymerized sea cucumber glycosaminoglycan;

FIG. 1-1 is a purity diagram of natural molecular segments of sea cucumber glycosaminoglycan;

FIG. 1-2 is a purity diagram of depolymerized sea cucumber glycosaminoglycan with a weight average molecular weight of 54,876 Da;

FIG. 1-3 is a purity diagram of depolymerized sea cucumber glycosaminoglycan with a weight average molecular weight of 60,915 Da;

FIG. 1-4 is a purity diagram of depolymerized sea cucumber glycosaminoglycan with a weight average molecular weight of 64,904 Da;

FIG. 1-5 is a purity diagram of depolymerized sea cucumber glycosaminoglycan with a weight average molecular weight of 71,147 Da;

FIG. 1-6 is a purity diagram of depolymerized sea cucumber glycosaminoglycan with a weight average molecular weight of 74,844 Da;

FIG. 1-7 is a purity diagram of depolymerized sea cucumber glycosaminoglycan with a weight average molecular weight of 80,336 Da;

FIG. 1-8 is a purity diagram of depolymerized sea cucumber glycosaminoglycan with a weight average molecular weight of 84,481 Da;

FIG. 1-9 is a purity diagram of depolymerized sea cucumber glycosaminoglycan with a weight average molecular weight of 90,919 Da;

FIG. 1-10 is a purity diagram of depolymerized sea cucumber glycosaminoglycan with a weight average molecular weight of 95,821 Da;

FIG. 1-11 is a purity diagram of depolymerized sea cucumber glycosaminoglycan with a weight average molecular weight of 10,1250 Da;

FIG. 1-12 is a purity diagram of depolymerized sea cucumber glycosaminoglycan with a weight average molecular weight of 10,3998 Da;

FIG. 1-13 is a purity diagram of depolymerized sea cucumber glycosaminoglycan with a weight average molecular weight of 10,9161 Da;

FIG. 1-14 is a purity diagram of depolymerized sea cucumber glycosaminoglycan with a weight average molecular weight of 115,268 Da;

FIG. 1-15 is a purity diagram of depolymerized sea cucumber glycosaminoglycan with a weight average molecular weight of 121,017 Da.

FIG. 2 is a test report of molecular weight of sea cucumber glycosaminoglycan and depolymerized sea cucumber glycosaminoglycan;

FIG. 2-1 is a test report of molecular weight of natural molecular segments of sea cucumber glycosaminoglycan;

FIG. 2-2 is a test report of molecular weight of depolymerized sea cucumber glycosaminoglycan with a weight average molecular weight of 54,876 Da;

FIG. 2-3 is a test report of molecular weight of depolymerized sea cucumber glycosaminoglycan with a weight average molecular weight of 60,915 Da;

FIG. 2-4 is a test report of molecular weight of depolymerized sea cucumber glycosaminoglycan with a weight average molecular weight of 64,904 Da;

FIG. 2-5 is a test report of molecular weight of depolymerized sea cucumber glycosaminoglycan with a weight average molecular weight of 71,147 Da;

FIG. 2-6 is a test report of molecular weight of depolymerized sea cucumber glycosaminoglycan with a weight average molecular weight of 74,844 Da;

FIG. 2-7 is a test report of molecular weight of depolymerized sea cucumber glycosaminoglycan with a weight average molecular weight of 80,336 Da;

FIG. 2-8 is a test report of molecular weight of depolymerized sea cucumber glycosaminoglycan with a weight average molecular weight of 84,481 Da;

FIG. 2-9 is a test report of molecular weight of depolymerized sea cucumber glycosaminoglycan with a weight average molecular weight of 90,919 Da;

FIG. 2-10 is a test report of molecular weight of depolymerized sea cucumber glycosaminoglycan with a weight average molecular weight of 95,821 Da;

FIG. 2-11 is a test report of molecular weight of depolymerized sea cucumber glycosaminoglycan with a weight average molecular weight of 10,1250 Da;

FIG. 2-12 is a test report of molecular weight of depolymerized sea cucumber glycosaminoglycan with a weight average molecular weight of 10,3998 Da;

FIG. 2-13 is a test report of molecular weight of depolymerized sea cucumber glycosaminoglycan with a weight average molecular weight of 10,9161 Da;

FIG. 2-14 is a test report of molecular weight of depolymerized sea cucumber glycosaminoglycan with a weight average molecular weight of 115,268 Da;

FIG. 2-15 is a test report of molecular weight of depolymerized sea cucumber glycosaminoglycan with a weight average molecular weight of 121,017 Da.

FIG. 3 is a diagram of a linear relationship between the in vitro anticoagulant dose of depolymerized sea cucumber glycosaminoglycan and the blood coagulation time;

FIG. 3-1 is the result of in vitro anticoagulation experiments of DHG-1;

FIG. 3-2 is the result of in vitro anticoagulation experiments of DHG-2.

### DETAILED DESCRIPTION OF THE PREFERRED EMBODIMENTS

An extraction method of depolymerized sea cucumber glycosaminoglycan refers to steps of: extracting sea cucumber glycosaminoglycan from sea cucumbers, degrading and depolymerizing to generate depolymerized glycosaminoglycan, and collecting the depolymerized sea cucumber glycosaminoglycan with the desired molecular weight. The method for extracting the sea cucumber glycosaminoglycan from the body wall of the sea cucumbers is familiar to those of skill in the art, such as the Chinese patent ZL200910305363.5.

### Example 1

### Extraction of Sea Cucumber Glycosaminoglycan

4.5 kg of a medicinal material of holothuria leucospilota was weighed, and soaked with water for 16 h. The body wall of the sea cucumber was drained, minced, weighed and replenished with water to 36 kg, and placed into a 60° C. water bath, into which was added 6 mol/L sodium hydroxide to adjust pH to 8.2±0.2. 90 ml of a proteolytic enzyme Alcalase (Novozymes (Shenyang) Biotechnology Co., Ltd.) was added therein and stirred, to carry out enzymolysis for 4 h. The mixture was inactivated for 10 min at a temperature above 85° C., and cooled to 49° C.±2° C. 6 mol/L sodium hydroxide was added therein to adjust pH to 8.2±0.2, followed by the addition of 9 g of a compound pancreatin under a brand of Xuemei from Wuxi City Xuemei Enzyme Formulation Science and Technology Co., Ltd. The mixture was stirred to carry out enzymolysis for 4 h, boiled for 15 min, cooled, and centrifugated at 5° C., to collect a supernatant. 6 mol/L hydrochloric acid was added therein to adjust pH to 2.5±0.2. The mixture was refrigerated for 2 h at 4° C., and centrifugated, to collect a supernatant. 6 mol/L sodium hydroxide was added therein to adjust pH to 7.5±0.2. 0.8 time volume of ethanol was added therein, and the mixture was stood still for 12 h at 4° C.

The mixture was centrifugated, and a precipitate was collected and weighed, into which was added 8 times weight of distilled water, and the resulting mixture was heated to 85° C.±2° C. After complete dissolution, 6 mol/L sodium hydroxide was added therein to adjust pH to 9.0±0.2, and calcium chloride was added, to a calcium chloride concentration in the solution up to 3% (w/v). The temperature was rised to 92° C. and maintained for 15 min, then cooled to room temperature, and the mixture was centrifugated at 4° C., to collect a supernatant. A saturated sodium carbonate solution was used to adjust pH to 11.0±0.1, and the mixture was centrifugated, to collect a supernatant. 6 mol/L hydrochloric acid was used to adjust pH to 6.0±0.1. 1 time volume of ethanol was added therein, and the mixture was refrigerated for 12 h at 4° C.

The refrigerated liquid was centrifugated, and a precipitate was collected and weighed, into which was added 2 times volume of distilled water. The mixture was heated to sufficiently dissolve. Potassium acetate was added therein to allow it to have a final concentration of 2 mol/L. The mixture was stood still for 12 h at 4° C., and centrifugated. A precipitate was collected and weighed, into which was added 2 times volume of distilled water. The resulting mixture was heated to sufficiently dissolve. Potassium acetate was added therein to allow it to have a final concentration of 2 mol/L. The mixture was stood still for 12 h at 4° C., and centrifugated. The precipitate was washed with a cold 2 mol/L potassium acetate solution three times, and then washed with 80% ethanol, 95% ethanol, and anhydrous ethanol, successively. After ethanol was volatiled to depletion, the precipitate was dried at 80° C. and weighed, so as to obtain a crude product A.

The crude product A was dissolved with a 0.05 mol/L pH 6.0 HAc-NaAc buffer solution to prepare a 2% solution for column packing. After passing through a cellulose chromatographic column, the solution was washed with 1.5 times column volumes of an HAc-NaAc buffer solution (pH 6.0±0.1) with 0.4 mol/L NaCl, and then eluted with an HAc-NaAc buffer solution (pH 6.0±0.1) with 1 mol/L NaCl. An eluate was collected according to the value change rate at 220 nm with an UV detector, placed into a 60° C. water bath, and adjusted to pH 11±0.1 with NaOH, and 3% hydrogen peroxide by volume was added therein. The mixture was held for 4 h, cooled, and centrifugated, to collect a supernatant. HCl was used to adjust pH to 7.2±0.1. 1 time volume of ethanol was added therein, and the mixture was stood still for 12 h at 4° C.

The mixture was centrifugated, and a precipitate was collected and washed with 80% ethanol, 95% ethanol, and anhydrous ethanol successively, so as to obtain a crude product B.

The crude product B was dissolved with distilled water into a 5% solution, concentrated with an ultrafiltration membrane with molecular weight cut off of 10,000 to ½ of the original volume, replenished with water to the original volume, and ultrafiltered again to ½ of the volume. Water was added again to repeat once the above steps, and an ultrafiltrate was freeze dried, so as to obtain the sea cucumber glycosaminoglycan.

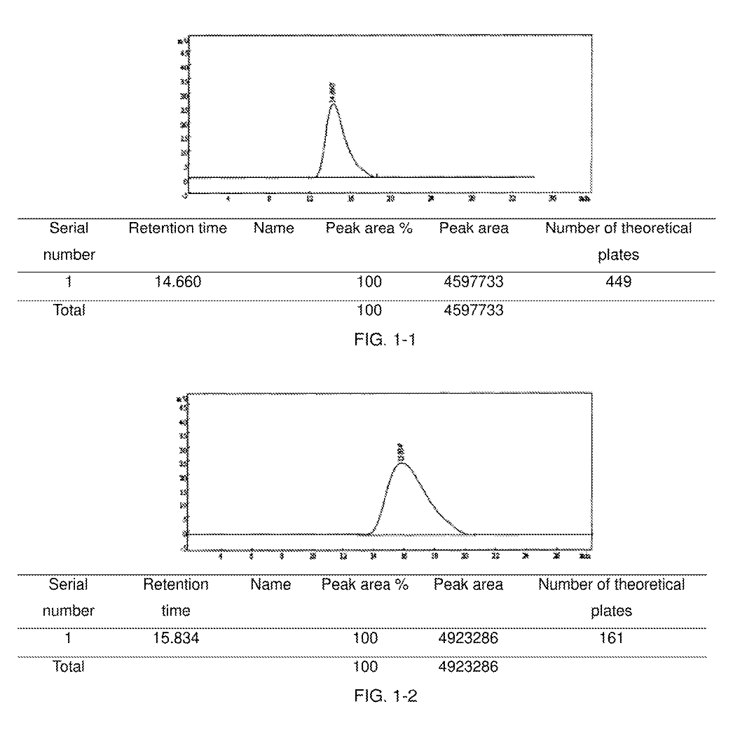

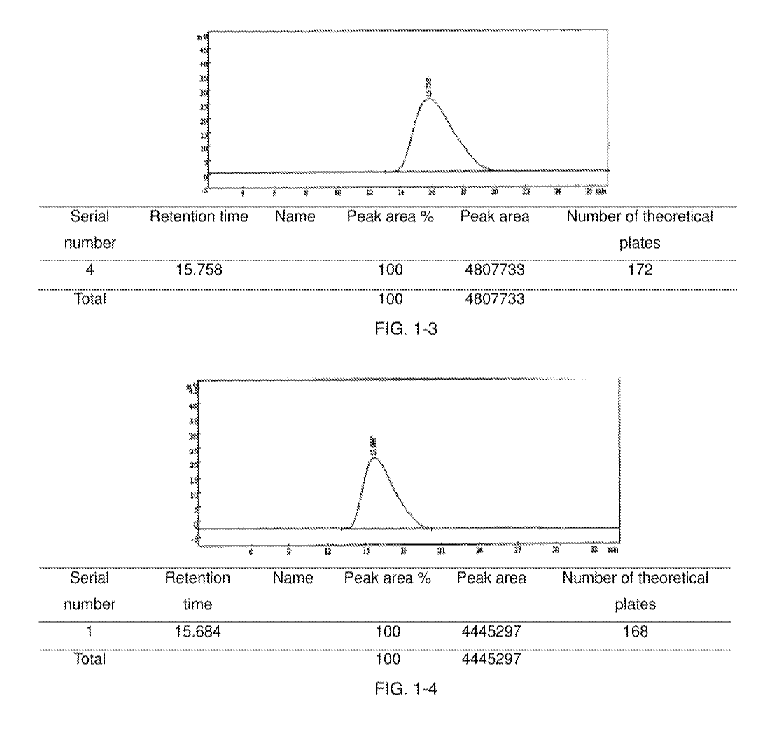

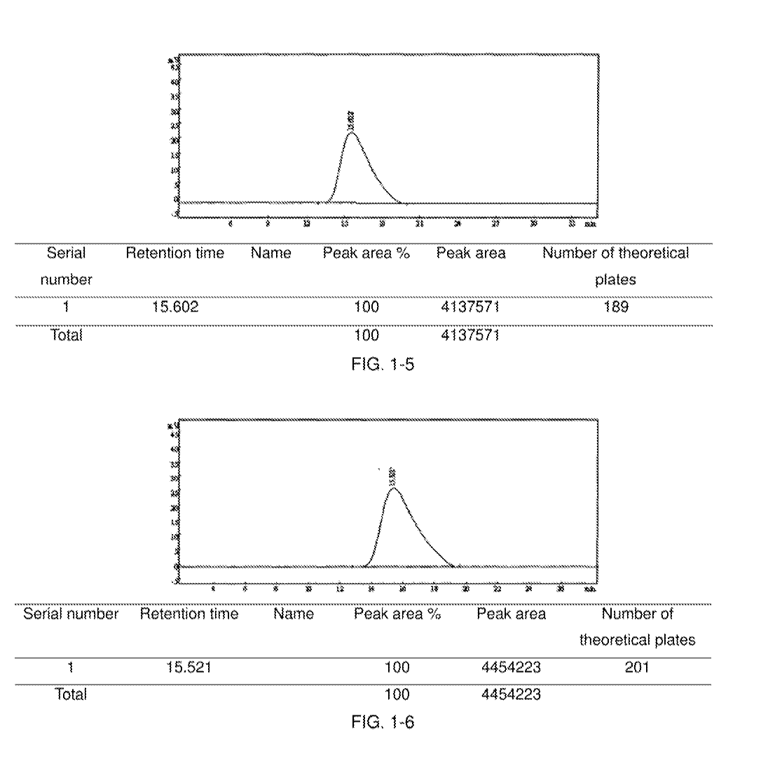

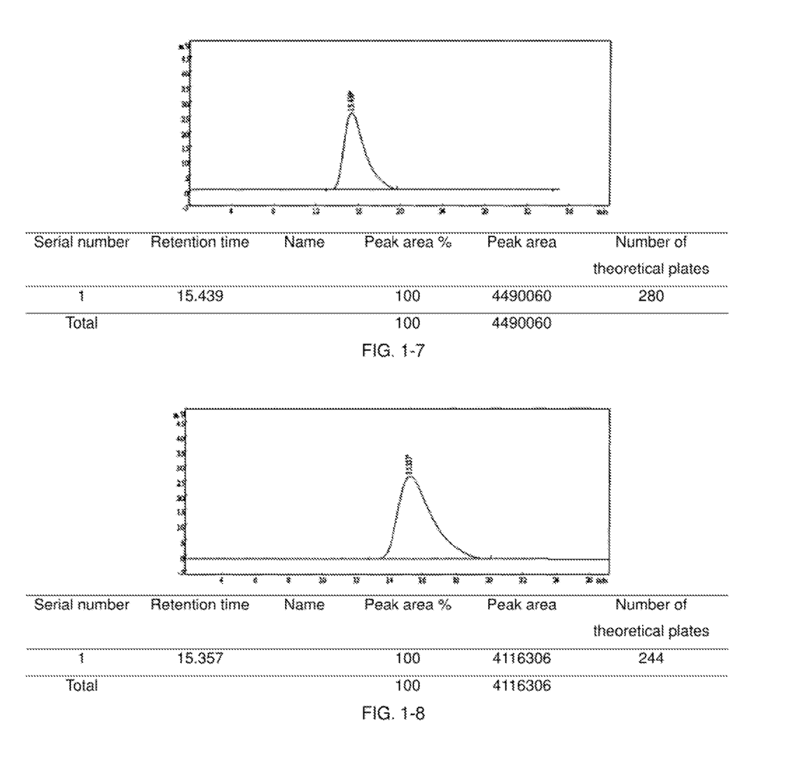

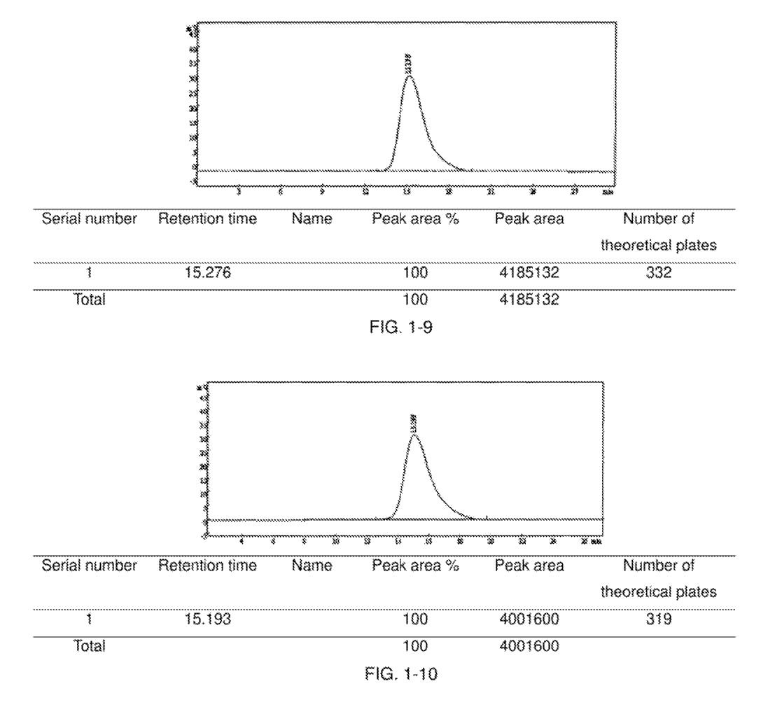

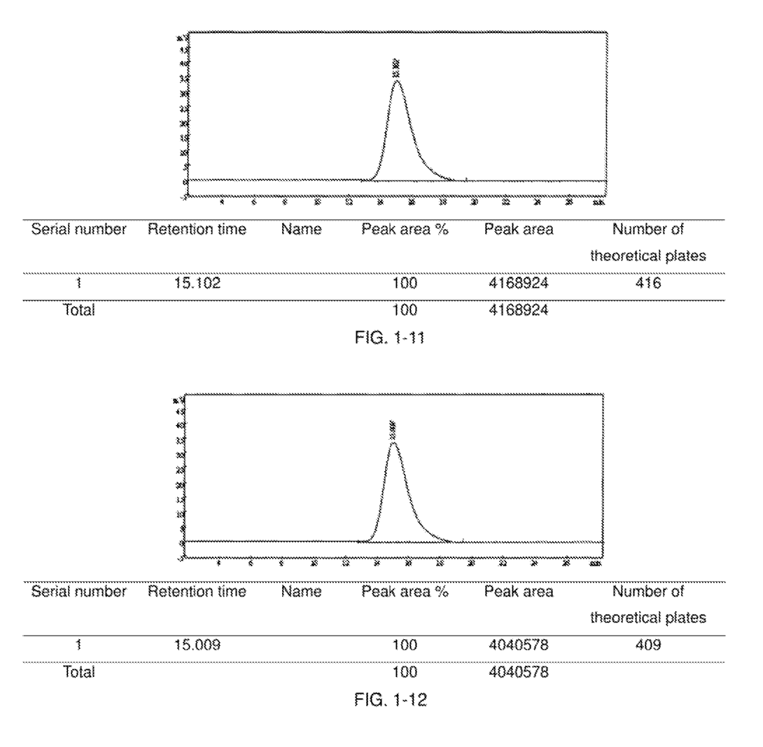

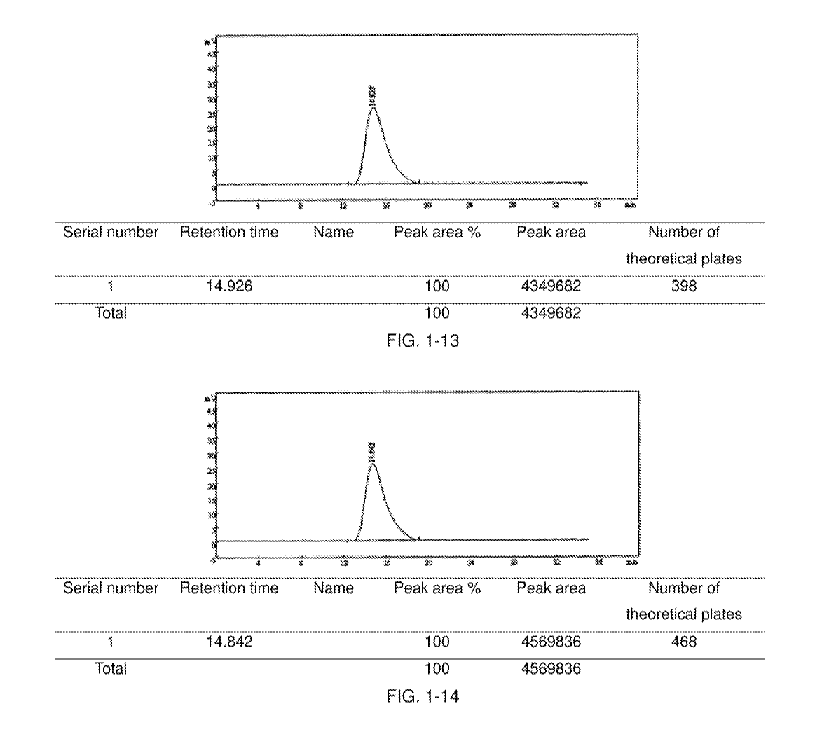

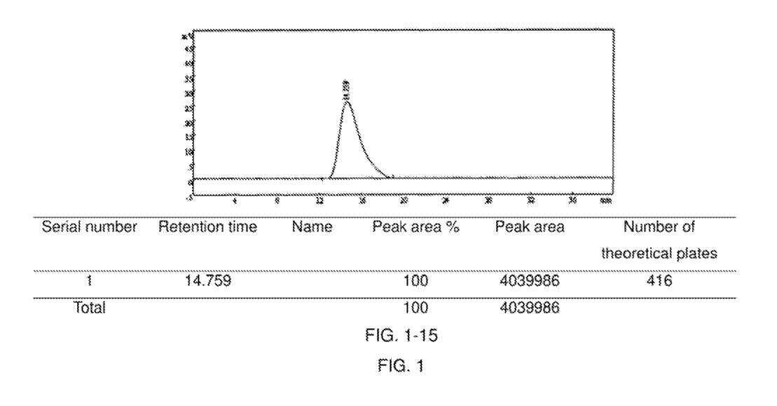

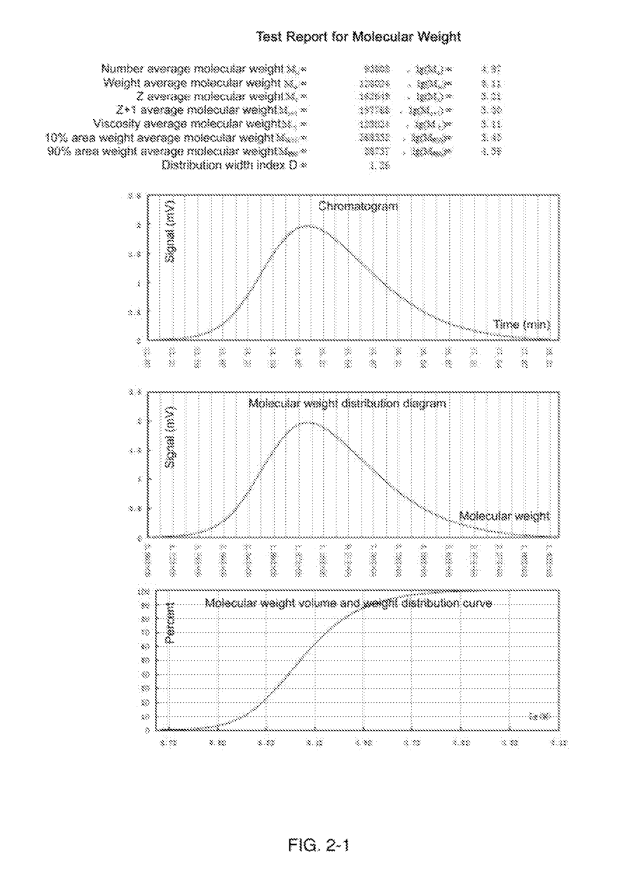

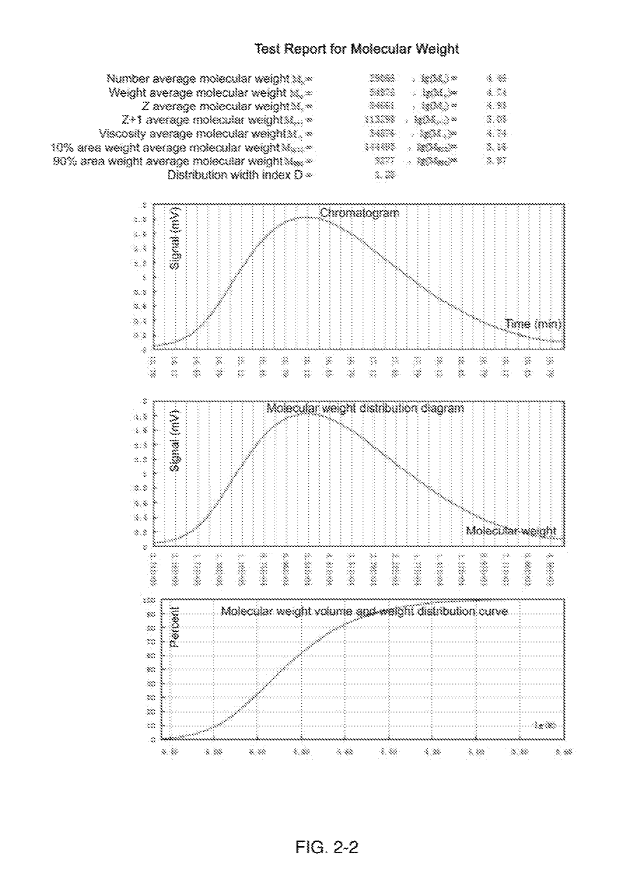

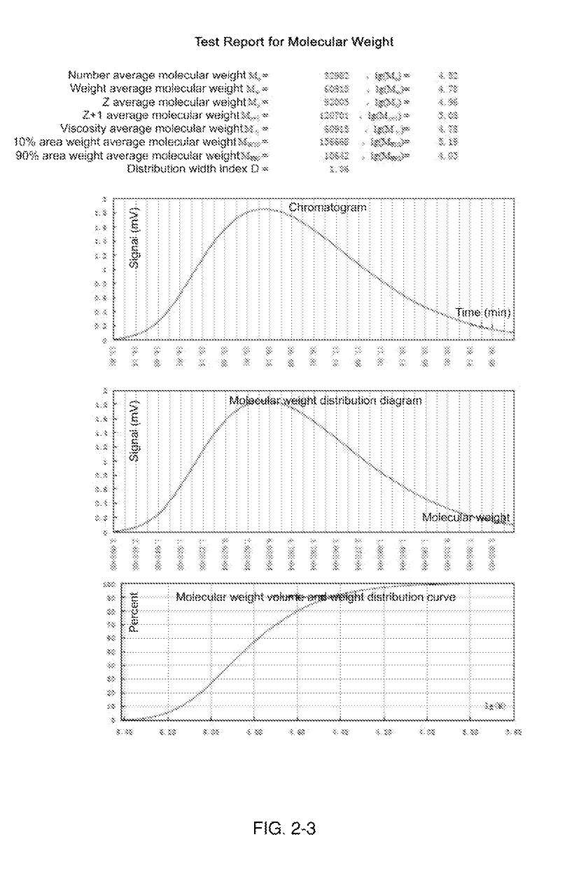

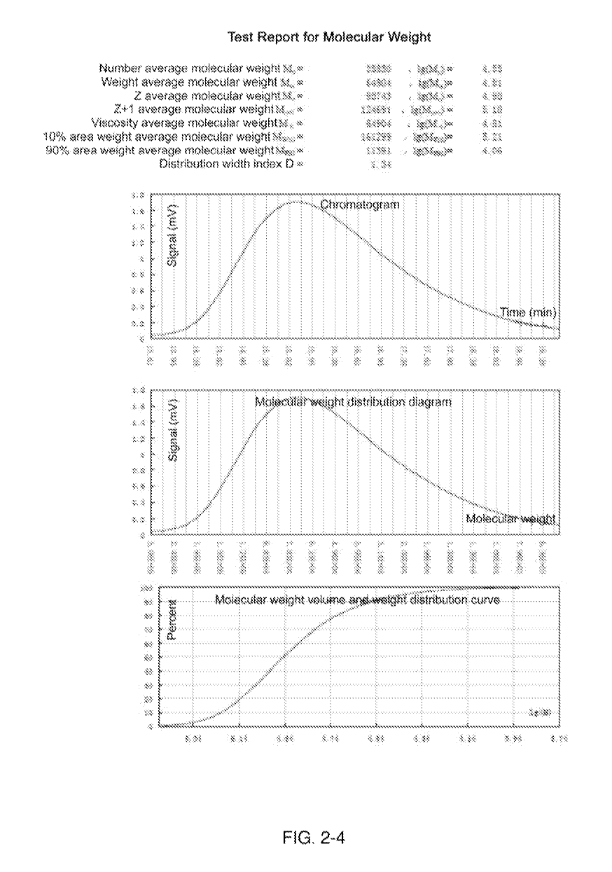

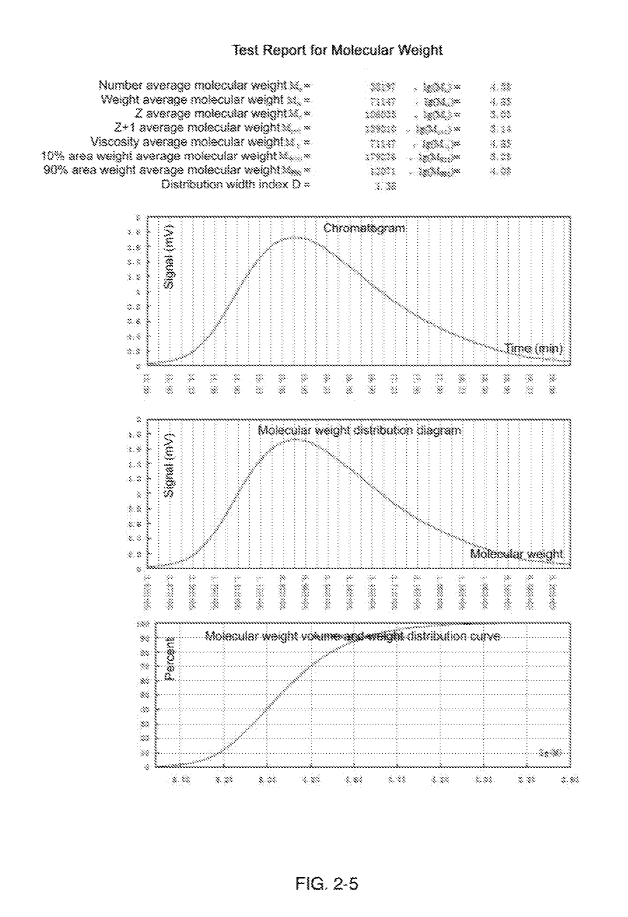

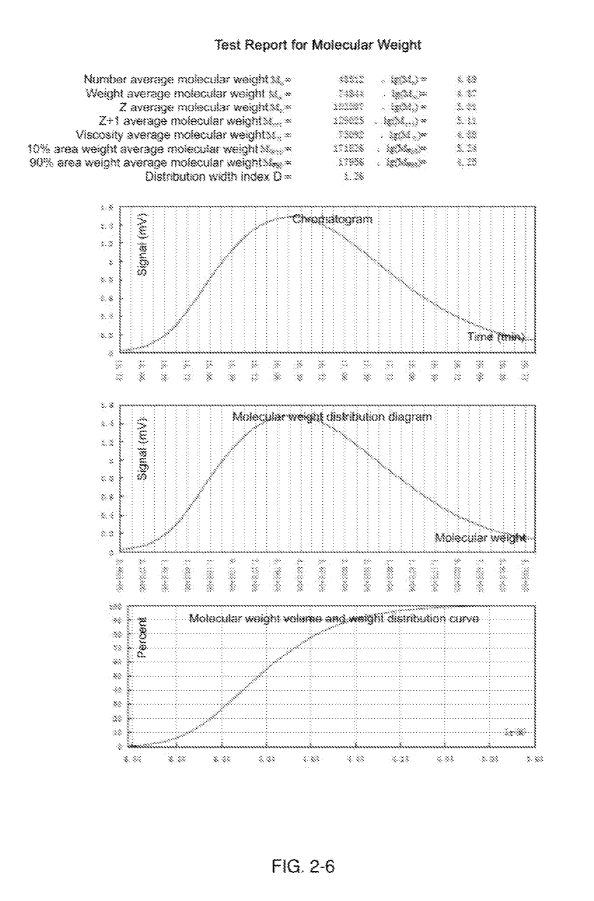

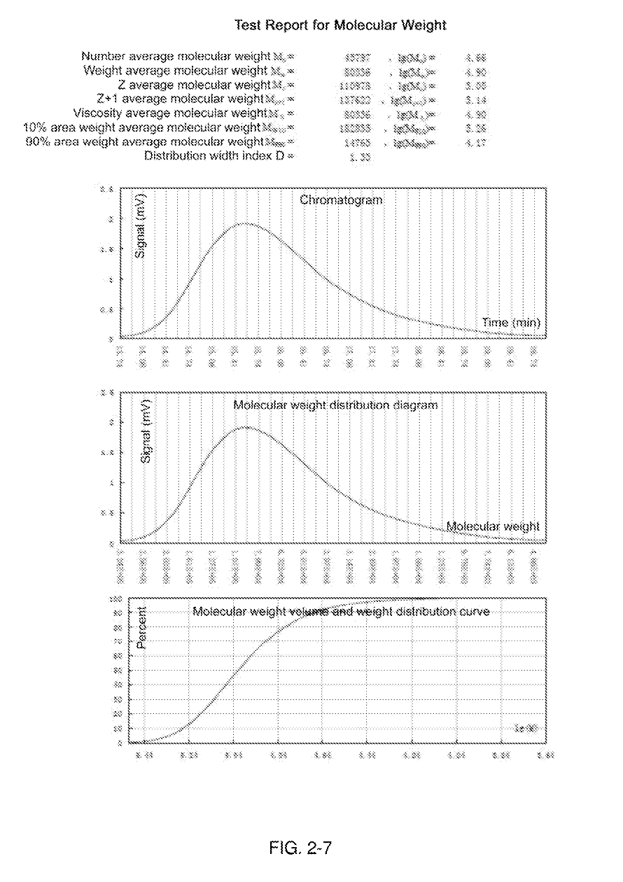

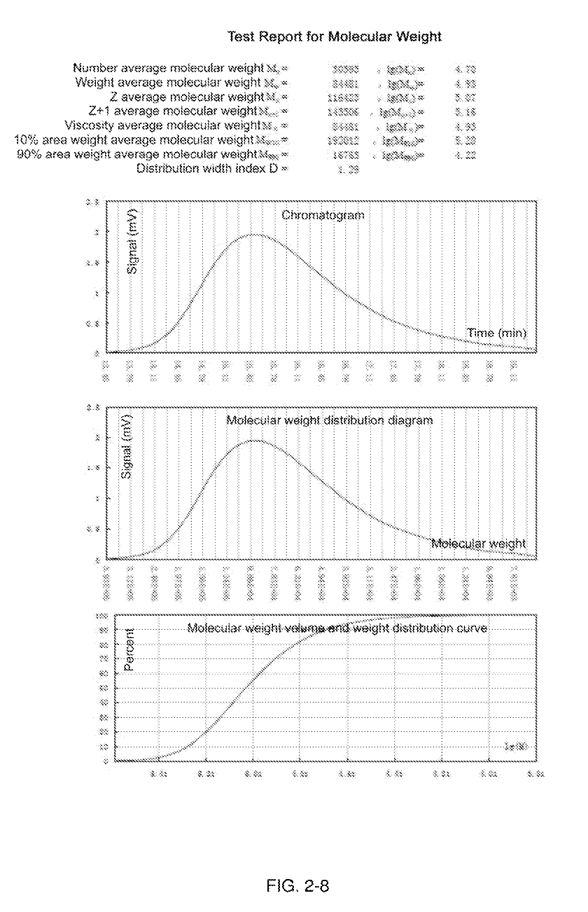

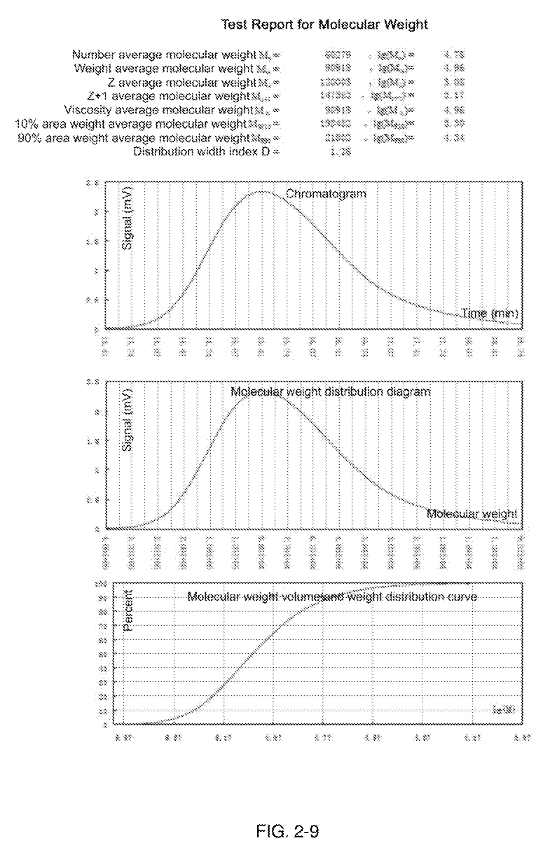

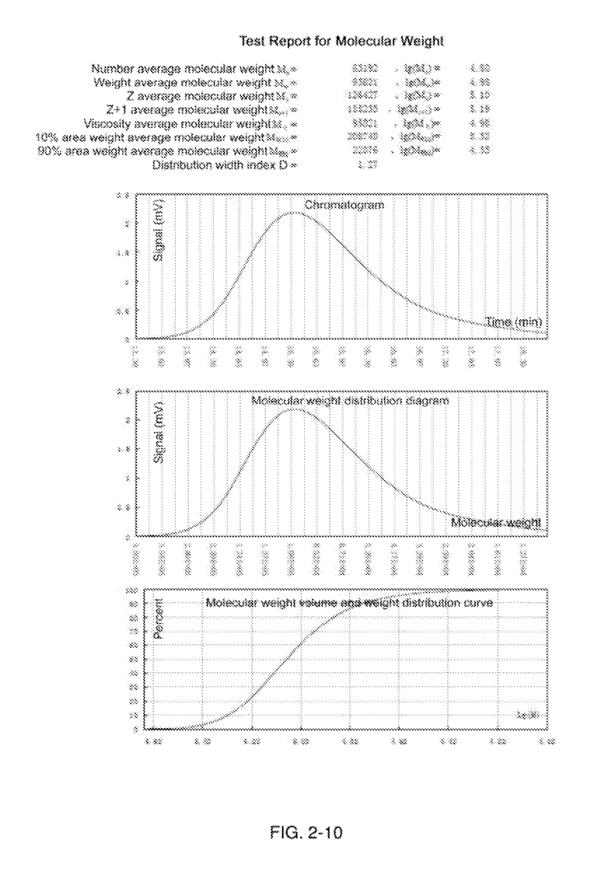

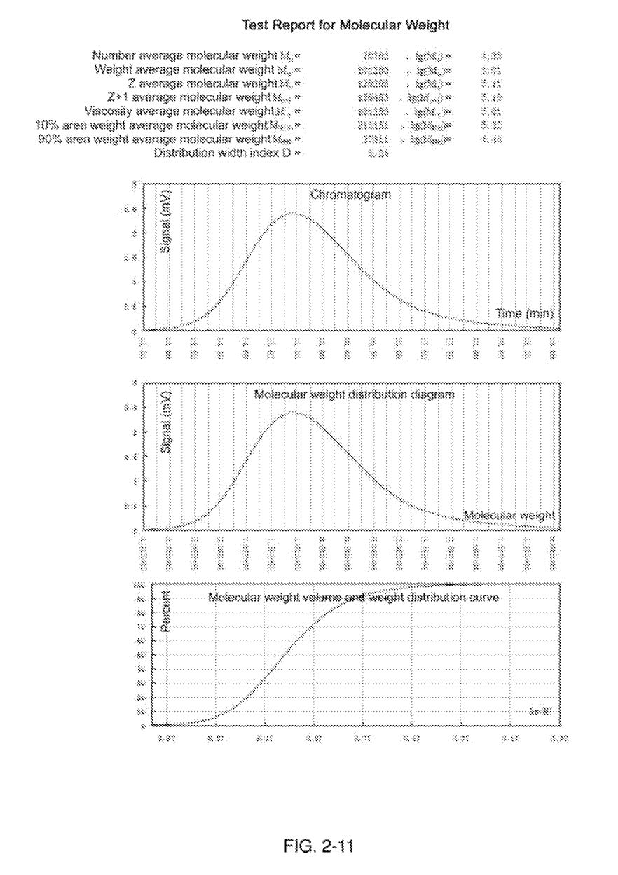

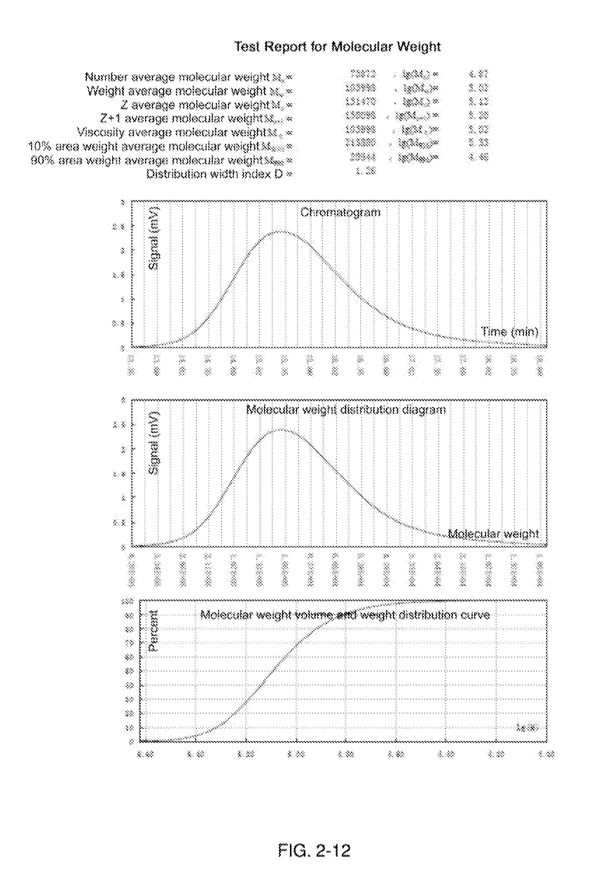

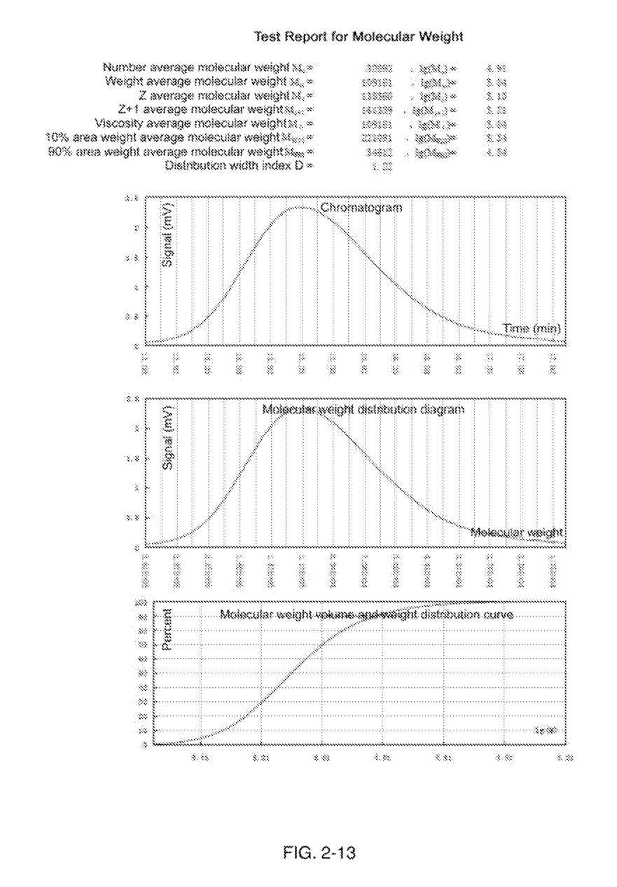

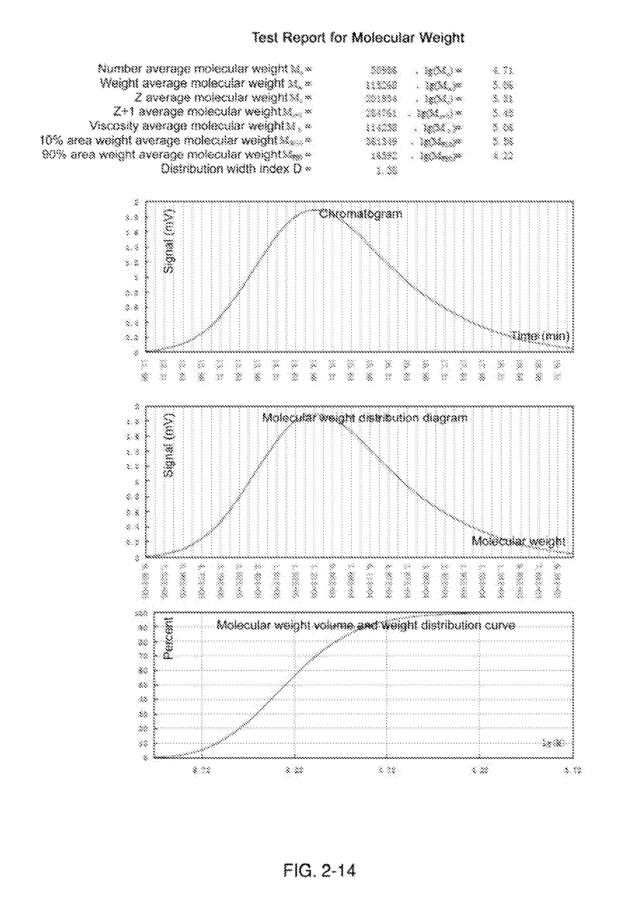

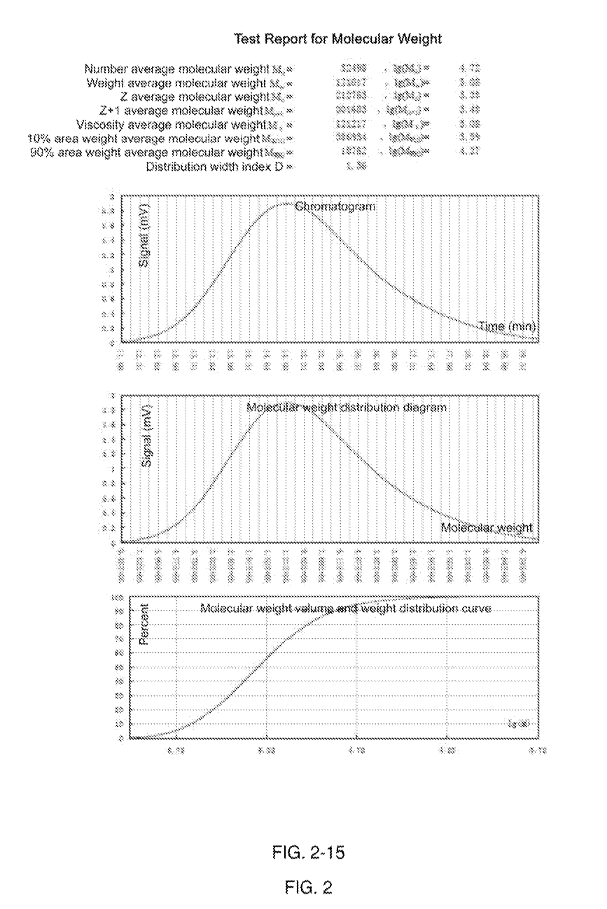

The sea cucumber glycosaminoglycan obtained in this example was subjected to a differential refractive index detector (RID-10A, Shimadzu) to obtain a pure product with a purity of 99.0% (the chromatogram is seen in FIG. 1-1). The depolymerized sea cucumber glycosaminoglycan obtained in this example was subjected to a gel column (TSK gel G4000PWXL, TOSOH) for chromatographic analysis, to know that the weight average molecular weight of the product was 128,024 Da, and the D value was 1.26 (the chromatogram is seen in FIG. 2-1).

### Example 2

### Preparation of Depolymerized Sea Cucumber Glycosaminoglycan

### Example 2-1

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 4 h and 50 min at 40° C. The solution was neutralized to be neutral with 0.1 mol/1 sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 54,500 Da and 57,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-2). The depolymerized sea cucumber glycosaminoglycan obtained in this example was subjected to a gel column (TSK gel G4000PWXL, TOSOH) for chromatographic analysis, to know that the weight average molecular weight of the product was 54,876 Da, and the D value was 1.28 (the chromatogram is seen in FIG. 2-2).

### Example 2-2

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 4 h and 20 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 58,000 Da and 62,000 Da, a D value<1.5, and a purity higher than 98%.

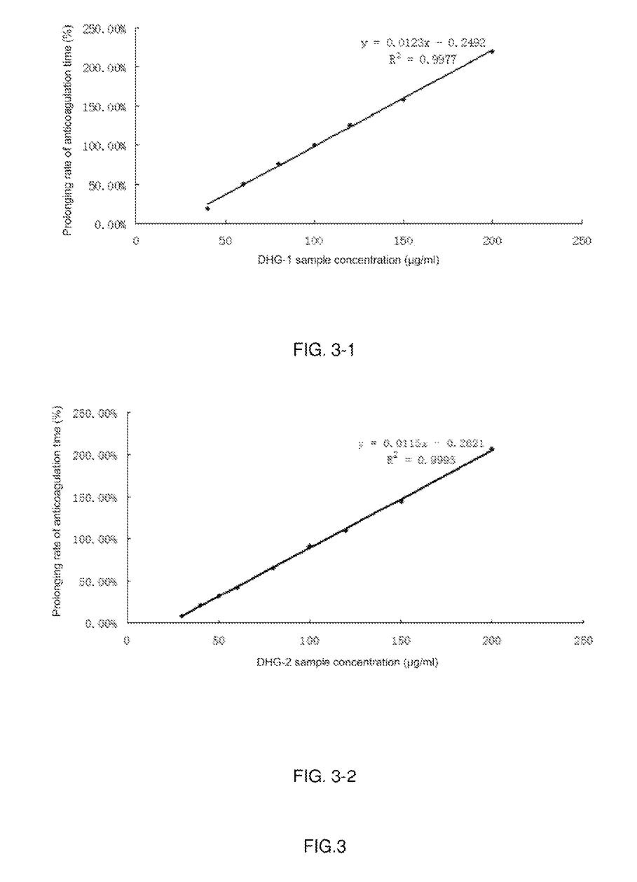

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-3). The depolymerized sea cucumber glycosaminoglycan obtained in this example was subjected to a gel column (TSK gel G4000PWXL, TOSOH) for chromatographic analysis, to know that the weight average molecular weight of the product was 60,915 Da, and the D value was 1.36 (the chromatogram is seen in FIG. 2-3).

### Example 2-3

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 3 h and 50 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 63,000 Da and 67,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-4). The depolymerized sea cucumber glycosaminoglycan obtained in this example was subjected to a gel column (TSK gel G4000PWXL, TOSOH) for chromatographic analysis, to know that the weight average molecular weight of the product was 64,904 Da, and the D value was 1.34 (the chromatogram is seen in FIG. 2-4).

### Example 2-4

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 3 h and 20 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 68,000 Da and 72,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-5). The depolymerized sea cucumber glycosaminoglycan obtained in this example was subjected to a gel column (TSK gel G4000PWXL, TOSOH) for chromatographic analysis, to know that the weight average molecular weight of the product was 71,147 Da, and the D value was 1.38 (the chromatogram is seen in FIG. 2-5).

### Example 2-5

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 2 h and 55 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 73,000 Da and 77,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-6). The depolymerized sea cucumber glycosaminoglycan obtained in this example was subjected to a gel column (TSK gel G4000PWXL, TOSOH) for chromatographic analysis, to know that the weight average molecular weight of the product was 74,844 Da, and the D value was 1.26 (the chromatogram is seen in FIG. 2-6).

### Example 2-6

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 2 h and 30 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 78,000 Da and 82,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-7). The depolymerized sea cucumber glycosaminoglycan obtained in this example was subjected to a gel column (TSK gel G4000PWXL, TOSOH) for chromatographic analysis, to know that the weight average molecular weight of the product was 80,336 Da, and the D value was 1.33 (the chromatogram is seen in FIG. 2-7).

### Example 2-7

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 2 h and 5 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 83,000 Da and 87,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-8). The depolymerized sea cucumber glycosaminoglycan obtained in this example was subjected to a gel column (TSK gel G4000PWXL, TOSOH) for chromatographic analysis, to know that the weight average molecular weight of the product was 84,481 Da, and the D value was 1.29 (the chromatogram is seen in FIG. 2-8).

### Example 2-8

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 1 h and 40 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 88,000 Da and 92,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-9). The depolymerized sea cucumber glycosaminoglycan obtained in this example was subjected to a gel column (TSK gel G4000PWXL, TOSOH) for chromatographic analysis, to know that the weight average molecular weight of the product was 90,919 Da, and the D value was 1.26 (the chromatogram is seen in FIG. 2-9).

### Example 2-9

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 1 h and 15 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 93,000 Da and 97,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-10). The depolymerized sea cucumber glycosaminoglycan obtained in this example was subjected to a gel column (TSK gel G4000PWXL, TOSOH) for chromatographic analysis, to know that the weight average molecular weight of the product was 95,821 Da, and the D value was 1.27 (the chromatogram is seen in FIG. 2-10).

### Example 2-10

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 55 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 98,000 Da and 102,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-11). The depolymerized sea cucumber glycosaminoglycan obtained in this example was subjected to a gel column (TSK gel G4000PWXL, TOSOH) for chromatographic analysis, to know that the weight average molecular weight of the product was 101,250 Da, and the D value was 1.24 (the chromatogram is seen in FIG. 2-11).

### Example 2-11

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 40 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 103,000 Da and 107,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-12). The depolymerized sea cucumber glycosaminoglycan obtained in this example was subjected to a gel column (TSK gel G4000PWXL, TOSOH) for chromatographic analysis, to know that the weight average molecular weight of the product was 103,998 Da, and the D value was 1.26 (the chromatogram is seen in FIG. 2-12).

### Example 2-12

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 30 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 108,000 Da and 112,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-13). The depolymerized sea cucumber glycosaminoglycan obtained in this example was subjected to a gel column (TSK gel G4000PWXL, TOSOH) for chromatographic analysis, to know that the weight average molecular weight of the product was 109,161 Da, and the D value was 1.22 (the chromatogram is seen in FIG. 2-13).

### Example 2-13

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 20 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 113,000 Da and 117,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-14). The depolymerized sea cucumber glycosaminoglycan obtained in this example was subjected to a gel column (TSK gel G4000PWXL, TOSOH) for chromatographic analysis, to know that the weight average molecular weight of the product was 115,268 Da, and the D value was 1.38 (the chromatogram is seen in FIG. 2-14).

### Example 2-14

The pure product of sea cucumber glycosaminoglycan from the above Example 1 was prepared into a 5% solution with 5% acetic acid. 30% hydrogen peroxide was added therein so that the concentration of hydrogen peroxide in the solution was 3%, and controlled depolymerization was carried out for 10 min at 40° C. The solution was neutralized to be neutral with 0.1 mol/l sodium hydroxide, 3 times volume of ethanol was added for alcohol precipitation, and the resultant mixture was stood still and centrifugated, to obtain a crude product of depolymerized sea cucumber glycosaminoglycan.

The crude product was dried and dissolved in 5 times weight of water, subjected to a sephadex-G75 column and eluted with 0.5 mol/l sodium chloride to remove salts and low molecular impurities, and the desalted sample was freeze dried to obtain 55 g of depolymerized sea cucumber glycosaminoglycan with molecular weights all between 118,000 Da and 122,000 Da, a D value<1.5, and a purity higher than 98%.

The depolymerized sea cucumber glycosaminoglycan obtained in this example was subjected to a differential refractive index detector (RID-10A, Shimadzu) to obtain a pure product with a purity of 99.0% (the chromatogram is seen in FIG. 1-15). The depolymerized sea cucumber glycosaminoglycan obtained in this example was subjected to a gel column (TSK gel G4000PWXL, TOSOH) for chromatographic analysis, to know that the weight average molecular weight of the product was 121,017 Da, and the D value was 1.36 (the chromatogram is seen in FIG. 2-15).

### Example 3

40.0 g of sea cucumber glycosaminoglycan or depolymerized sea cucumber glycosaminoglycan obtained above was added into 80 g of mannitol, and 1000 ml of water for injection was added therein for dissolution. The solution was ultrafiltered, encapsulated, and lyophilized, to obtain 1000 bottles of lyophilized injection powders of sea cucumber glycosaminoglycan or depolymerized sea cucumber glycosaminoglycan for injection.

### Example 4

Pharmacodynamic experiments of sea cucumber glycosaminoglycan and depolymerized sea cucumber glycosaminoglycan

4.1 In Vitro Anticoagulation Experiment

4.1.1 Test Materials

Test Samples:

Name: depolymerized sea cucumber glycosaminoglycan, abbreviated as follows: DHG; DHG-1 (Example 2-1) and DHG-2 (Example 2-6); formulation: normal saline for injection was used to dilute the glycosaminoglycan to a desired concentration after precise suction.

Test Animals

Strain: rabbit; source: Shanghai Chenhang experimental rabbit Co. Ltd.; gender: male; weight: 1850 g; animal certificate number: SCXK (Shanghai) 2008-0010.

4.1.2 Test Instrument

Platelet aggregation and coagulation factor analyzer (Model: LG-PABER Beijing Steellex Scientific Instrument Company).

4.1.3 Experimental Method

On the experimental day, 80 μl of rabbit plasma and 10 μl of a 0.9% sodium chloride solution were respectively added into sample pools, and preheated for 180 s, and then 10 μl of a 1% calcium chloride solution was added therein to be uniformly mixed at once to avoid the generation of air bubbles. The platelet aggregation and coagulation factor analyzer was used to start calculating time, and the coagulation time of each sample pool was recorded, i.e., a blank group.

A control solution was precisely weighed, diluted with a 0.9% sodium chloride solution to solutions of different concentrations, i.e., sample solutions DHG-1 (40.0 μg/ml to 200.0 μg/ml) and DHG-2 (30.0 μg/ml to 200.0 μg/ml) of different concentrations.

10 μl of sample solutions of different concentrations were used in place of 10 μl of the 0.9% sodium chloride solution to respectively determine the plasma coagulation time of the sample solution of each concentration. The parallel determination was carried out 4 times for each concentration, and an average value was given.

4.1.4 Experimental Results

Experimental results showed that the final concentrations of the samples were in dosage ranges of DHG-1 (40.0 μg/ml to 200.0 μg/ml) and DHG-2 (30.0 μg/ml to 200.0 μg/ml), the blood coagulation time was prolonged as the dosage increased, in an alleviating trend. Therefore, the depolymerized sea cucumber glycosaminoglycan composition has better safety and controllability in anticoagulation.

TABLE 1-1
Results of in vitro anticoagulation experiment of DHG-1

| DHG-1 (μg/ml) | Blood coagulation time (s) | Prolongation rate (%) |
|---|---|---|
| Blank | 290.8 ± 10.5 | |
| 40 | 345.1 ± 11.3 | 18.68% |
| 60 | 437.6 ± 14.2 | 50.49% |
| 80 | 512.4 ± 13.4 | 76.22% |
| 100 | 582.9 ± 15.6 | 100.46% |
| 120 | 656.5 ± 17.1 | 125.78% |
| 150 | 749.5 ± 18.4 | 157.76% |
| 200 | 928.9 ± 23.3 | 219.46% |

TABLE 1-2
Results of in vitro anticoagulation experiment of DHG-2

| DHG-2 (μg/ml) | Blood coagulation time (s) | Prolongation rate (%) |
|---|---|---|
| Blank | 277.9 ± 11.7 | |
| 30 | 302.2 ± 11.2 | 8.38% |
| 40 | 338.6 ± 13.5 | 20.93% |
| 50 | 372.5 ± 12.8 | 32.61% |
| 60 | 399.1 ± 15.6 | 41.78% |
| 80 | 468.2 ± 12.7 | 65.60% |
| 100 | 542.6 ± 14.4 | 91.25% |
| 120 | 596.8 ± 17.1 | 109.94% |
| 150 | 698.1 ± 16.3 | 144.86% |
| 200 | 878.2 ± 19.2 | 206.94% |

4.2 Effect of Subcutaneously Injected Depolymerized Sea Cucumber Glycosaminoglycan and Natural Sea Cucumber Glycosaminoglycan on Rat Blood Coagulation System

4.2.1 Test Materials

Test Samples:

Name: natural sea cucumber glycosaminoglycan, low molecular weight sea cucumber glycosaminoglycan (54,876 Da, 60,915 Da, 74,844 Da, and 90,919 Da); formulation: normal saline for injection was used to dilute the glycosaminoglycan to a desired concentration after precise suction.

4.2.2 Test Animals

Strains: SD rats; source: Shanghai Super—B&K experimental animal Co, Ltd.; gender: male; weight: 180-200 g; animal certificate number: SCXK (Shanghai) 2008-0016; breeding: animals were bred in purifying positive pressure ventilation animal rooms at a room temperature of 23±1° C., and a humidity of 50 to 70%, the artificial lighting simulated diurnal variation, and the animals had free access to food and water.

4.2.3 Test Instrument

Automatic Coagulation Analyzer Sysmex CA-1500

4.2.4 Experimental Method

10 SD rats each were assigned to a dosage group and a negative control group (subcutaneously injected with 0.5 ml of normal saline). Two dosage groups (10 and 20 mg/kg) were dosed through subcutaneous injection in a volume of 0.5 ml. At different periods of time (0.5 h, 1.0 h, 2.0 h, 4 h, 6 h, 8 h, and 12 h) after the subcutaneous injection, the numerical values of the prothrombin time (PT), the activated partial thromboplastin time (APTT) and the thrombin time (TT) were determined by collecting blood from the abdominal aorta. See Tables 2 and 4.

At 10 min before a surgery, the animals in each group were subjected to intraperitoneal injection of 3% Seconal to be anesthetized (0.1 ml/100 g body weight), and were supinely fixed to undergo an abdominal surgery, and the blood was collected by a disposable 3.2% sodium citrate anticoagulant vacuum blood collection tube.

4.2.5 Test Results

The depolymerized sea cucumber glycosaminoglycan and natural sea cucumber glycosaminoglycan at 10 mg/kg and 20 mg/kg had evident effects on APTT, TT, and PT. Depolymerized sea cucumber glycosaminoglycan with different weight average molecular weights had progressively increasing anticoagulant activity as the time increases progressively. Anticoagulant prolonging rate reached a peak value at time between 2 and 8 h, and depolymerized sea cucumber glycosaminoglycan with a lower weight average molecular weight reached a peak value earlier than that with a higher weight average molecular weight. The anticoagulant effect generated by subcutaneous injection of the natural molecular segments of sea cucumber glycosaminoglycan reached a peak value at 6 h at a dose of 10 mg/kg; and reached a peak value at 6 h at a dose of 20 mg/kg. Different molecular segments of the depolymerized sea cucumber glycosaminoglycan had different onset time of action as well as time to the peak value of the action at doses of 10 mg/kg and 20 mg/kg. Subcutaneously injected depolymerized sea cucumber glycosaminoglycan had an extremely significant effect on APTT, allowing APTT to have extension beyond the range of 150% to 250%. See Tables 2-1, 2-2, 2-3, and 2-4.

TABLE 2-1
Results of anticoagulation experiment of subcutaneous injection of DHG in rats

| | Molecular | | | | | | | |
|---|---|---|---|---|---|---|---|---|
| | segment | Blood coagulation time (s) | | | | | | |
| Item | (10 mg/kg) | 0.5 h | 1.0 h | 2 h | 4 h | 6 h | 8 h | 12 h |
| APTT | 54,876 Da | 22.3 ± 1.1 | 28.1 ± 1.4 | 35.3 ± 0.9 | 34.5 ± 0.6 | 21.9 ± 0.7 | 20.1 ± 0.9 | 17.4 ± 0.3 |
| | 60,915 Da | 21.5 ± 0.9 | 22.6 ± 0.5 | 29.0 ± 0.7 | 31.9 ± 0.8 | 25.1 ± 0.4 | 22.4 ± 0.6 | 17.7 ± 0.4 |
| | 74,844 Da | 18.7 ± 0.5 | 21.5 ± 1.0 | 26.3 ± 0.6 | 27.6 ± 0.7 | 21.6 ± 0.5 | 20.2 ± 0.6 | 17.4 ± 0.5 |
| | 90,919 Da | 17.1 ± 0.7 | 19.0 ± 0.4 | 21.9 ± 0.5 | 24.7 ± 0.8 | 20.7 ± 0.7 | 19.7 ± 0.3 | 17.3 ± 0.7 |
| | Natural | 15.3 ± 0.5 | 15.6 ± 0.6 | 17.3 ± 0.3 | 18.9 ± 0.4 | 21.2 ± 0.9 | 19.5 ± 0.4 | 16.6 ± 0.6 |
| | Blank | 14.3 ± 0.6 | 14.0 ± 0.8 | 15.0 ± 0.4 | 15.3 ± 0.4 | 14.9 ± 0.4 | 15.5 ± 0.4 | 15.1 ± 0.4 |
| PT | 54,876 Da | 16.5 ± 0.4 | 17.4 ± 0.6 | 19.3 ± 0.5 | 18.5 ± 0.8 | 17.0 ± 0.3 | 16.3 ± 0.5 | 15.7 ± 0.5 |
| | 60,915 Da | 16.2 ± 0.6 | 16.3 ± 0.4 | 17.5 ± 0.5 | 17.9 ± 0.5 | 17.7 ± 0.7 | 17.1 ± 0.6 | 15.9 ± 0.6 |
| | 74,844 Da | 15.9 ± 0.5 | 16.1 ± 0.4 | 17.1 ± 0.7 | 17.4 ± 0.6 | 16.7 ± 0.5 | 16.2 ± 0.5 | 15.6 ± 0.4 |
| | 90,919 Da | 15.7 ± 0.4 | 15.9 ± 0.3 | 16.5 ± 0.5 | 16.6 ± 0.7 | 16.5 ± 0.5 | 16.0 ± 0.4 | 15.6 ± 0.2 |
| | Natural | 15.4 ± 0.1 | 15.5 ± 0.7 | 15.9 ± 0.4 | 16.0 ± 0.9 | 16.6 ± 0.5 | 16.2 ± 0.7 | 15.5 ± 0.3 |
| | Blank | 15.1 ± 0.4 | 14.9 ± 0.3 | 15.2 ± 0.2 | 15.0 ± 0.4 | 15.3 ± 0.1 | 15.2 ± 0.3 | 15.0 ± 0.7 |
| TT | 54,876 Da | 46.4 ± 1.2 | 50.7 ± 0.7 | 56.7 ± 1.5 | 54.8 ± 1.7 | 48.2 ± 0.9 | 44.6 ± 0.8 | 43.8 ± 0.7 |
| | 60,915 Da | 45.6 ± 0.4 | 47.7 ± 0.8 | 53.3 ± 0.6 | 53.5 ± 1.2 | 48.8 ± 0.5 | 45.0 ± 0.6 | 44.3 ± 0.9 |
| | 74,844 Da | 43.3 ± 1.1 | 45.5 ± 1.4 | 49.6 ± 0.7 | 52.1 ± 0.9 | 47.3 ± 0.5 | 44.3 ± 0.4 | 43.7 ± 0.6 |
| | 90,919 Da | 41.6 ± 0.4 | 43.5 ± 0.7 | 47.5 ± 0.5 | 47.9 ± 0.8 | 44.7 ± 0.5 | 43.5 ± 0.6 | 42.9 ± 1.3 |
| | Natural | 39.9 ± 1.2 | 42.3 ± 0.6 | 45.6 ± 0.8 | 45.7 ± 0.5 | 46.1 ± 0.9 | 44.7 ± 0.4 | 42.8 ± 0.5 |
| | Blank | 39.1 ± 1.2 | 40.2 ± 0.3 | 42.5 ± 0.6 | 41.7 ± 0.5 | 40.8 ± 0.6 | 40.5 ± 0.8 | 41.2 ± 0.4 |

TABLE 2-2
Blood coagulation time prolonging rate of DHG in rats

| | Molecular | | | | | | | |
|---|---|---|---|---|---|---|---|---|
| | segment | Time prolonging rate (%) | | | | | | |
| Item | (10 mg/kg) | 0.5 h | 1.0 h | 2 h | 4 h | 6 h | 8 h | 12 h |
| APTT | 54,876 Da | 56.10% | 100.69% | 135.28% | 125.71% | 47.22% | 29.89% | 14.99% |
| | 60,915 Da | 50.69% | 61.50% | 93.35% | 108.56% | 68.52% | 44.32% | 17.02% |
| | 74,844 Da | 30.52% | 53.88% | 75.00% | 80.45% | 45.19% | 30.02% | 15.38% |
| | 90,919 Da | 19.46% | 35.42% | 45.68% | 61.32% | 39.25% | 27.31% | 14.35% |
| | Natural | 7.02% | 11.21% | 15.02% | 23.45% | 42.10% | 25.98% | 10.23% |
| PT | 54,876 Da | 8.96% | 16.52% | 27.20% | 23.31% | 11.25% | 7.04% | 4.65% |
| | 60,915 Da | 7.31% | 9.68% | 15.43% | 19.04% | 15.83% | 12.74% | 5.69% |
| | 74,844 Da | 5.37% | 7.79% | 12.31% | 16.18% | 9.24% | 6.87% | 4.21% |
| | 90,919 Da | 4.01% | 6.86% | 8.37% | 10.89% | 7.64% | 5.34% | 3.81% |
| | Natural | 1.97% | 3.87% | 4.84% | 6.69% | 8.62% | 6.48% | 3.17% |
| TT | 54,876 Da | 18.64% | 26.09% | 33.52% | 31.52% | 18.02% | 10.21% | 6.42% |
| | 60,915 Da | 16.55% | 18.67% | 25.48% | 28.24% | 19.50% | 11.02% | 7.61% |
| | 74,844 Da | 10.64% | 13.09% | 16.77% | 24.98% | 15.85% | 9.36% | 6.01% |
| | 90,919 Da | 6.35% | 8.21% | 11.68% | 14.83% | 9.57% | 7.32% | 4.23% |
| | Natural | 2.10% | 5.32% | 7.21% | 9.52% | 13.08% | 10.27% | 3.97% |

TABLE 2-3
Results of anticoagulation experiment of subcutaneous injection of DHG in rats

| | Molecular | Blood coagulation time (s) | | | | | | |
|---|---|---|---|---|---|---|---|---|
| Item | segment | 0.5 h | 1.0 h | 2 h | 4 h | 6 h | 8 h | 12 h |
| | (20 mg/kg) | | | | | | | |
| APTT | 54,876 Da | 35.3 ± 1.0 | 42.1 ± 0.7 | 51.6 ± 0.8 | 60.6 ± 1.6 | 54.8 ± 0.5 | 50.0 ± 1.4 | 37.6 ± 0.6 |
| | 60,915 Da | 30.6 ± 0.7 | 38.4 ± 0.6 | 45.0 ± 0.5 | 58.4 ± 0.9 | 64.2 ± 1.9 | 59.7 ± 1.3 | 42.3 ± 0.5 |
| | 74,844 Da | 28.5 ± 0.4 | 31.3 ± 0.3 | 42.2 ± 0.4 | 54.3 ± 1.7 | 61.5 ± 1.3 | 55.9 ± 1.2 | 39.6 ± 1.3 |
| | 90,919 Da | 23.2 ± 0.3 | 28.4 ± 0.9 | 29.9 ± 0.7 | 38.2 ± 1.1 | 42.1 ± 0.8 | 38.0 ± 0.9 | 27.7 ± 0.7 |
| | Natural | 17.3 ± 0.5 | 18.4 ± 0.4 | 19.7 ± 0.3 | 24.0 ± 0.3 | 29.3 ± 0.4 | 26.6 ± 0.3 | 21.4 ± 0.3 |
| | Blank | 15.0 ± 0.4 | 15.2 ± 0.0 | 14.9 ± 0.3 | 15.1 ± 0.1 | 15.0 ± 0.3 | 15.3 ± 0.2 | 14.9 ± 0.1 |
| PT | 54,876 Da | 18.0 ± 0.5 | 18.7 ± 0.4 | 19.2 ± 0.6 | 20.0 ± 0.6 | 19.6 ± 0.4 | 18.5 ± 0.5 | 18.1 ± 0.4 |
| | 60,915 Da | 17.2 ± 0.3 | 18.0 ± 0.1 | 18.7 ± 0.5 | 19.8 ± 0.7 | 20.7 ± 0.8 | 19.5 ± 0.7 | 18.7 ± 0.6 |
| | 74,844 Da | 16.6 ± 0.4 | 17.7 ± 0.5 | 18.4 ± 0.6 | 19.7 ± 0.8 | 20.5 ± 0.7 | 19.2 ± 0.5 | 17.5 ± 0.3 |
| | 90,919 Da | 16.0 ± 0.1 | 16.6 ± 0.6 | 17.0 ± 0.4 | 17.9 ± 0.3 | 18.9 ± 0.6 | 17.5 ± 0.4 | 16.3 ± 0.5 |
| | Natural | 15.4 ± 0.4 | 15.8 ± 0.2 | 15.7 ± 0.3 | 16.3 ± 0.4 | 17.3 ± 0.6 | 16.2 ± 0.6 | 16.0 ± 0.8 |
| | Blank | 14.9 ± 0.1 | 15.1 ± 0.3 | 14.8 ± 0.4 | 15.0 ± 0.5 | 15.2 ± 0.1 | 14.8 ± 0.2 | 14.9 ± 0.3 |
| | (10 mg/kg) | | | | | | | |
| TT | 54,876 Da | 60.4 ± 0.9 | 61.6 ± 1.4 | 68.5 ± 1.8 | 73.1 ± 1.9 | 68.8 ± 1.6 | 66.9 ± 1.7 | 61.6 ± 1.5 |
| | 60,915 Da | 58.7 ± 1.4 | 60.4 ± 1.2 | 64.5 ± 1.5 | 70.3 ± 0.9 | 74.1 ± 0.7 | 72.6 ± 1.1 | 63.2 ± 1.8 |
| | 74,844 Da | 57.1 ± 1.3 | 58.8 ± 1.8 | 63.4 ± 1.6 | 69.9 ± 1.5 | 72.2 ± 1.5 | 67.0 ± 1.7 | 60.2 ± 1.6 |
| | 90,919 Da | 52.4 ± 1.5 | 55.3 ± 1.7 | 59.2 ± 0.9 | 62.5 ± 1.3 | 62.5 ± 1.4 | 62.0 ± 1.6 | 55.8 ± 1.2 |
| | Natural | 43.5 ± 0.9 | 46.4 ± 1.4 | 49.7 ± 1.3 | 54.1 ± 0.6 | 56.6 ± 1.7 | 56.1 ± 1.4 | 52.0 ± 1.5 |
| | Blank | 39.5 ± 1.1 | 39.1 ± 0.9 | 40.0 ± 0.7 | 40.3 ± 0.8 | 39.5 ± 0.6 | 40.4 ± 1.0 | 39.8 ± 1.1 |

TABLE 2-4
Blood coagulation time prolonging rate of DHG in rats

| | Molecular | | | | | | | |
|---|---|---|---|---|---|---|---|---|
| | segment | Time prolonging rate (%) | | | | | | |
| Item | (20 mg/kg) | 0.5 h | 1.0 h | 2 h | 4 h | 6 h | 8 h | 12 h |
| APTT | 54,876 Da | 135.15% | 176.39% | 245.68% | 301.25% | 265.31% | 226.51% | 152.10% |
| | 60,915 Da | 103.57% | 152.30% | 201.54% | 286.52% | 327.71% | 289.65% | 183.54% |
| | 74,844 Da | 89.60% | 105.70% | 182.84% | 259.48% | 309.86% | 265.14% | 165.37% |
| | 90,919 Da | 54.62% | 86.59% | 100.52% | 152.65% | 180.23% | 148.36% | 85.42% |
| | Natural | 14.97% | 20.54% | 31.97% | 58.74% | 94.93% | 73.21% | 43.56% |
| PT | 54,876 Da | 20.42% | 23.56% | 29.68% | 33.29% | 28.51% | 24.85% | 21.24% |
| | 60,915 Da | 15.24% | 18.67% | 26.02% | 31.83% | 36.03% | 31.62% | 24.97% |
| | 74,844 Da | 11.05% | 16.87% | 24.28% | 30.81% | 34.28% | 29.65% | 17.32% |
| | 90,919 Da | 7.26% | 9.65% | 14.85% | 18.79% | 24.21% | 17.68% | 9.08% |
| | Natural | 3.24% | 4.56% | 6.07% | 8.23% | 13.25% | 8.96% | 7.21% |
| TT | 54,876 Da | 52.88% | 57.35% | 71.20% | 81.34% | 73.93% | 65.37% | 54.69% |
| | 60,915 Da | 48.36% | 54.27% | 61.04% | 74.21% | 87.55% | 79.62% | 58.78% |
| | 74,844 Da | 44.31% | 50.31% | 58.39% | 73.22% | 82.55% | 65.77% | 51.25% |
| | 90,919 Da | 32.62% | 41.41% | 47.90% | 55.02% | 58.11% | 53.28% | 40.05% |
| | Natural | 9.98% | 18.66% | 24.16% | 34.20% | 43.26% | 38.68% | 30.47% |

4.3 Effect of Depolymerized Sea Cucumber Glycosaminoglycan on Rat Arteriovenous Catheter Thrombosis Model

4.3.1 Test Materials

Test Samples:

Name: depolymerized sea cucumber glycosaminoglycan (DHG); formulation: normal saline for injection was used to dilute the glycosaminoglycan to a desired concentration after precise suction. Control Sample: name: heparin; source: Sinopharm Chemical Reagent Co., Ltd.; batch number: F20091029; content: 150 U/mg; formulation: normal saline for injection was used to dissolve and dilute the glycosaminoglycan to a desired concentration after precise weighing.

Test Animals: strains: SD rats; source: Shanghai Super—B&K experimental animal Co., Ltd.; gender: male; weight: 180 to 220 g; animal certificate number: SCXK (Shanghai) 2008-0016; breeding: animals were bred in purifying positive pressure ventilation animal rooms at a room temperature of 23±1° C., and a humidity of 50 to 70%, the artificial lighting simulated diurnal variation, and the animals had free access to food and water.

4.3.2 Test Instrument

BS 110 s-type electronic balance, produced by SARTORIUS Corporation, with a minimum weight value of 0.1 mg.

4.3.3 Test Method

10 SD rats each were assigned to different dosage groups, a negative control group (normal saline 1 ml/kg) and a positive control low molecular weight heparin group (2 mg/kg). All drugs were dosed through subcutaneous injection in a volume of 0.5 ml.

The animals in each group were subjected to intraperitoneal injection of 12% chloral hydrate to be anesthetized (350 to 400 mg/kg) 10 min before a surgery, and then were supinely fixed. The neck skin was cut off, and the left carotid artery and the right external jugular vein were dissected to be connected by a bypass pipe in which a 7-cm long No. 4 surgical silk thread was placed. The bloodstream was opened for 15 min at 2 h after the dosage respectively, and then the silk thread was taken out to be weighed, and the weight of the silk thread was deducted to obtain the wet weight of the thrombus. The mean and standard deviation of the wet weight of the thrombus in each test group were calculated and were compared with those of the normal saline group by a t-test. The inhibition rate of the wet weight of the thrombus in each test group was calculated in accordance with the following formula:

Inhibition rate of thrombus (%)=((Thrombus wet weight(solvent group)−Thrombus wet weight(test group))/Thrombus wet weight(solvent group)*100%

4.3.4 Test Results

See Table 3, both the positive drug and the test drug could obviously inhibit thrombus formation in the tests after the dosage. The inhibitory effects of the test drugs on thrombus formation were evident.

TABLE 3
Effect of DHG on rat arteriovenous catheter thrombosis model

| | | | Thrombosis | Thrombus inhibition |
|---|---|---|---|---|
| Group | n | Dose (mg/kg) | weight (mg) | rate (%) |
| Blank | 10 | 0.5 ml | 67.8 ± 3.6 | — |
| 54,876 Da | 10 | 20 | 36.4 ± 9.6** | 46.31% |
| 60,915 Da | 10 | 20 | 34.1 ± 7.8** | 49.71% |
| 74,844 Da | 9 | 20 | 35.2 ± 9.2** | 48.08% |
| 90,919 Da | 8 | 20 | 40.7 ± 10.4** | 39.97% |
| Natural | 10 | 20 | 48.9 ± 8.7** | 27.88% |

Comparison with the negative group: * P < 0.05, **P < 0.01

4.4 Effect of Subcutaneously Injected Depolymerized Sea Cucumber Glycosaminoglycan Composition with Different Molecular Weight Segments on Rat Blood Coagulation System

4.4.1 Test Materials

Test Samples:

Name: depolymerized sea cucumber glycosaminoglycan 54,876 Da, and 74,844 Da; natural sea cucumber glycosaminoglycan; source: Shanghai Kairun Bio-Medical Co., Ltd.; formulation: normal saline for injection was used to dilute the glycosaminoglycan to a desired concentration after precise suction.

4.4.2 Test Animals

Strains: SD rats; source: Shanghai Super—B&K experimental animal Co, Ltd.; gender: male; weight: 180-220 g; animal certificate number: SCXK (Shanghai) 2008-0016; breeding: animals were bred in purifying positive pressure ventilation animal rooms at a room temperature of 23±1° C., and a humidity of 50 to 70%, the artificial lighting simulated diurnal variation, and the animals had free access to food and water.

4.4.3 Test Instrument

Automatic Coagulation Analyzer Sysmex CA-1500

4.4.4 Experimental Method

10 SD rats each were assigned to different dosage groups, negative control groups (subcutaneously injected with 0.5 ml of normal saline). A composition of different molecular weight segments (54,876 Da, and 74,844 Da) of the depolymerized sea cucumber glycosaminoglycan and the natural sea cucumber glycosaminoglycan at a dose ratio of 1:1 (10 mg/kg) was dosed through subcutaneous injection. Normal saline for injection as a blank had a volume of 0.5 ml. At different periods of time after the subcutaneous injection, the numerical values of the activated partial thromboplastin time (APTT), the prothrombin time (PT), and the thrombin time (TT) were determined by collecting blood from the abdominal aorta. See Table 5.

At 10 min before a surgery, the animals in each group were subjected to intraperitoneal injection of 3% Seconal to be anesthetized (0.1 ml/100 g body weight), and were supinely fixed to undergo an abdominal surgery, and the blood was collected by a disposable 3.2% sodium citrate anticoagulant vacuum blood collection tube.

4.4.5 Test Results

Experimental results showed that, the subcutaneously injected depolymerized sea cucumber glycosaminoglycan composition with different molecular weight segments had significantly increased APTT and TT activity, and could overcome the defect in a mono-molecular weight segment of slow onset of action or short duration. Experimental data are seen in Tables 4-1 and 4-2.

TABLE 4-1
Results of anticoagulation experiments of subcutaneous injection
of different molecular segments of DHG in rats

| | Composition | Time (s) | | | | | | |
|---|---|---|---|---|---|---|---|---|
| Item | (20 mg/kg) | 0.5 h | 1.0 h | 2 h | 4 h | 6 h | 8 h | 12 h |
| APTT | 54,876 Da (10 | 31.3 ± 0.4 | 39.0 ± 0.6 | 48.3 ± 0.5 | 59.8 ± 0.7 | 61.4 ± 0.9 | 54.5 ± 0.6 | 39.0 ± 0.5 |
| | mg/kg) + 74,844 | | | | | | | |
| | Da (10 mg/kg) | | | | | | | |
| | 60,915 Da (10 | 24.9 ± 0.1 | 29.5 ± 0.3 | 36.4 ± 0.4 | 46.2 ± 0.5 | 57.5 ± 0.7 | 46.3 ± 0.4 | 34.3 ± 0.3 |
| | mg/kg) + natural | | | | | | | |
| | (10 mg/kg) | | | | | | | |
| | Blank | 15.1 ± 0.4 | 15 ± 0.5 | 14.9 ± 0.3 | 15.3 ± 0.6 | 15.2 ± 0.4 | 15.1 ± 0.1 | 15.0 ± 0.4 |
| PT | 54,876 Da (10 | 17.5 ± 0.3 | 17.8 ± 0.4 | 19.2 ± 0.7 | 19.7 ± 0.6 | 20.3 ± 0.9 | 19.5 ± 0.5 | 17.8 ± 0.4 |
| | mg/kg) + 74,844 | | | | | | | |
| | Da (10 mg/kg) | | | | | | | |
| | 60,915 Da (10 | 16.3 ± 0.4 | 16.9 ± 0.7 | 17.9 ± 0.5 | 18.8 ± 0.6 | 19.9 ± 0.8 | 19.0 ± 0.7 | 17.4 ± 0.6 |
| | mg/kg) + natural | | | | | | | |
| | (10 mg/kg) | | | | | | | |
| | Blank | 14.9 ± 0.1 | 14.8 ± 0.3 | 15.0 ± 0.4 | 14.9 ± 0.5 | 15.2 ± 0.4 | 14.9 ± 0.5 | 14.8 ± 0.2 |
| TT | 54,876 Da (10 | 60.3 ± 1.5 | 61.5 ± 1.2 | 65.5 ± 1.1 | 72.0 ± 1.7 | 74.0 ± 1.9 | 69.4 ± 1.0 | 61.7 ± 1.4 |
| | mg/kg) + 74,844 | | | | | | | |
| | Da (10 mg/kg) | | | | | | | |
| | 60,915 Da (10 | 55.6 ± 1.1 | 59.0 ± 1.4 | 59.9 ± 1.2 | 65.7 ± 1.5 | 70.7 ± 1.7 | 64.6 ± 1.3 | 59.3 ± 1.0 |
| | mg/kg) + natural | | | | | | | |
| | (10 mg/kg) | | | | | | | |
| | Blank | 40.1 ± 0.9 | 39.6 ± 0.7 | 39.1 ± 0.9 | 40.8 ± 1.0 | 40.5 ± 0.8 | 39.8 ± 0.7 | 39.7 ± 0.6 |

TABLE 4-2
Blood coagulation time prolonging rate of different molecular segments of DHG in rats

| | Composition | Time prolonging rate (%) | | | | | | |
|---|---|---|---|---|---|---|---|---|
| Items | (20 mg/kg) | 0.5 h | 1.0 h | 2 h | 4 h | 6 h | 8 h | 12 h |
| APTT | 54,876 Da (10 | 106.65% | 159.85% | 223.83% | 290.36% | 303.58% | 260.48% | 159.61% |
| | mg/kg) + 74,844 | | | | | | | |
| | Da (10 mg/kg) | | | | | | | |
| | 60,915 Da (10 | 64.56% | 96.25% | 143.82% | 201.41% | 278.11% | 206.51% | 128.37% |
| | mg/kg) + natural | | | | | | | |
| | (10 mg/kg) | | | | | | | |
| PT | 54,876 Da (10 | 16.95% | 20.23% | 27.64% | 32.15% | 33.47% | 30.51% | 19.94% |
| | mg/kg) + 74,844 | | | | | | | |
| | Da (10 mg/kg) | | | | | | | |
| | 60,915 Da (10 | 8.95% | 13.52% | 18.72% | 25.95% | 30.64% | 26.85% | 17.01% |
| | mg/kg) + natural | | | | | | | |
| | (10 mg/kg) | | | | | | | |
| TT | 54,876 Da (10 | 50.13% | 55.26% | 67.39% | 76.26% | 82.71% | 74.15% | 55.23% |
| | mg/kg) + 74,844 | | | | | | | |
| | Da (10 mg/kg) | | | | | | | |
| | 60,915 Da (10 | 38.52% | 48.79% | 52.95% | 60.87% | 74.52% | 62.28% | 49.35% |
| | mg/kg) + natural | | | | | | | |
| | (10 mg/kg) | | | | | | | |

1. A method of preventing or treating arterial thromboembolic disease, comprising administering one or more segments of depolymerized sea cucumber glycosaminoglycan or natural molecular segments of sea cucumber glycosaminoglycan having a weight average molecular weight greater than 54,500 Da to a patient in need thereof.

2. The method according to claim 1, wherein, the arterial thromboembolic disease is selected from the group consisting of: atherosclerotic thrombotic diseases, venous thromboembolic diseases, hypercoagulable states, thrombi formed after an operation, prevention of the formation of thrombi after an operation, and combinations thereof.

3. The method according to claim 1, wherein the depolymerized sea cucumber glycosaminoglycan or the natural molecular segments of sea cucumber glycosaminoglycan with a weight average molecular weight greater than 54,500 Da is selected from the group consisting of: a depolymerized sea cucumber glycosaminoglycan of any weight average molecular weight, a natural molecular segments of sea cucumber glycosaminoglycan with a weight average molecular weight greater than 54,500 Da, a multisegment mixture of the depolymerized sea cucumber glycosaminoglycan of any weight average molecular weight and natural molecular segments of sea cucumber glycosaminoglycan with a weight average molecular weight greater than 54,500 Da, and combinations thereof.

4. The method according to claim 1, wherein the depolymerized sea cucumber glycosaminoglycan has a weight average molecular weight selected from the group consisting of:
any segment of 54,500 Da to 57,000 Da, 57,010 Da to 62,990 Da, 63,000 Da to 67,000 Da, 67,010 Da to 72,990 Da, 73,000 Da to 77,000 Da, 77,010 Da to 82,990 Da, 83,000 Da to 87,000 Da, 87,010 Da to 92,990 Da, 93,000 Da to 97,000 Da, 97,010 Da to 102,900 Da, 103,000 Da to 107,000 Da, 107,010 Da to 112,990 Da, 113,000 Da to 117,900 Da, and 118,000 Da to 122,050 Da.

5. The method according to claim 1, wherein the depolymerized sea cucumber glycosaminoglycan has a weight average molecular weight selected from the group consisting of:
any segment of 54,500 Da to 57,000 Da, 58,000 Da to 62,000 Da, 63,000 Da to 67,000 Da, 68,000 Da to 72,000 Da, 73,000 Da to 77,000 Da, 78,000 Da to 82,000 Da, 83,000 Da to 87,000 Da, 88,000 Da to 92,000 Da, 93,000 Da to 97,000 Da, 98,000 Da to 102,000 Da, 103,000 Da to 107,000, 108,000 Da to 112,000 Da, 113,000 Da to 117,000 Da, and 118,000 Da to 122,000 Da.

6. The method according to claim 1, wherein the drug includes a therapeutically effective amount of the depolymerized sea cucumber glycosaminoglycan and a pharmaceutically acceptable carrier, and is an injection solution for administration through intravenous or subcutaneous injection, or a lyophilized injection powder.

7. The method according to claim 3, wherein the drug includes a therapeutically effective amount of the depolymerized sea cucumber glycosaminoglycan and a pharmaceutically acceptable carrier, and is an injection solution for administration through intravenous or subcutaneous injection, or a lyophilized injection powder.

8. The method according to claim 4, wherein the drug includes a therapeutically effective amount of the depolymerized sea cucumber glycosaminoglycan and a pharmaceutically acceptable carrier, and is an injection solution for administration through intravenous or subcutaneous injection, or a lyophilized injection powder.

9. The method according to claim 5, wherein the drug includes a therapeutically effective amount of the depolymerized sea cucumber glycosaminoglycan and a pharmaceutically acceptable carrier, and is an injection solution for administration through intravenous or subcutaneous injection, or a lyophilized injection powder.
